(19) United States
(12) Patent Application Publication
(10) Pub. No.: US 2024/0082238 A1
(43) Pub. Date: Mar. 14, 2024

(54) COMPOSITIONS AND METHODS FOR TREATING CANCER

(71) Applicant: UNIVERSITY OF IOWA RESEARCH FOUNDATION, Iowa City, IA (US)

(72) Inventors: Aliasger K. Salem, Iowa City, IA (US); Kareem Ebeid, Iowa City, IA (US); Youssef Wahib Naguib Ibrahim, Iowa City, IA (US); Sanjib Saha, Iowa City, IA (US); Emad I. Wafa, Iowa City, IA (US); Suhaila Alhaj-Suliman, Iowa City, IA (US)

(73) Assignee: UNIVERSITY OF IOWA RESEARCH FOUNDATION, Iowa City, IA (US)

(21) Appl. No.: 17/890,757

(22) Filed: Aug. 18, 2022

Related U.S. Application Data
(60) Provisional application No. 63/234,492, filed on Aug. 18, 2021.

(51) Int. Cl. A61K 31/495; A61K 9/16; A61K 31/337; A61P 15/00; A61P 35/00

(52) U.S. Cl. CPC A61K 31/495; A61K 9/1647; A61K 31/337; A61P 15/00; A61P 35/00

(57) ABSTRACT

The invention provides methods, compositions, and kits that are useful for treating cancer (e.g., endometrial cancer).

### CROSS-REFERENCE TO RELATED APPLICATION

This application claims priority to U.S. Provisional Application No. 63/234,492 that was filed on Aug. 18, 2021. The entire content of the application referenced above is hereby incorporated by reference herein.

### STATEMENT REGARDING FEDERALLY SPONSORED RESEARCH

This invention was made with government support under CA086862 awarded by the National Institutes of Health. The government has certain rights in the invention.

### BACKGROUND

Opposite to most other types of cancer among US women, endometrial cancer (EC) incidence and mortality rates are both rising (Lu K H, Broaddus R R. The New England Journal of Medicine. 2020; 383(21):2053-64, doi: 10.1056/NEJMra1514010) clearly in a positive correlation with obesity and other lifestyle-related risk factors (smoking, diabetes, parity, etc.) (Setiawan V W, et. al., Journal of Clinical Oncology, 2013; 31(20):2607-18, doi: 10.1200/JC-O.2012.48.2596; and Murali R, Soslow R A, Weigelt B., The Lancet Oncology, 2014; 15(7):e268-78, doi: 10.1016/S1470-2045(13)70591-6). EC is typically categorized into the more common type I and the less common but more aggressive type II, based on histological features, hormone dependence, prognosis, and overall clinical outcome. While type I EC is usually associated with a low incidence of lymphogenic metastasis, and low risk of relapse, type II EC is usually characterized by a high risk of metastasis and relapse, poor prognosis, and high mortality rates (Setiawan V W, et. al., Journal of Clinical Oncology, 2013; 31(20):2607-18, doi: 10.1200/JCO.2012.48.2596). Histologically, the estrogen-independent type II ECs mostly belong to serous type (3-10% of all ECs), while most type I ECs belong to endometrioid type (85% or more of all ECs) (Bell D W, Ellenson L H. Annual review of pathology. 2019; 14:339-67, doi: 10.1146/annurev-pathol-020117-043609; and Goebel E A, et. al., Virchows Archly. 2018; 472(6):885-96, doi: 10.1007/s00428-017-2279-8).

Compared to type I EC, type II EC is usually poorly differentiated and is mostly associated with deep myometrial invasion and atrophic endometrium (Bell D W, Ellenson L H. Annual review of pathology. 2019; 14:339-67, doi: 10.1146/annurev-pathol-020117-043609; Prat J, Gallardo et. al., Pathology. 2007; 39(1):72-87, doi: 10.1080/00313020601136153; and Setiawan V W, et. al., Journal of Clinical Oncology, 2013; 31(20):2607-18, doi: 10.1200/JC-O.2012.48.2596). While obesity is a major predisposing factor to which the increased incidence of EC is correlated (Amant F, Moerman et. al., Lancet. 2005; 366(9484):491-505, doi: 10.1016/S0140-6736(05)67063-8; and Setiawan V W, et. al., Journal of Clinical Oncology, 2013; 31(20):2607-18, doi: 10.1200/JCO.2012.48.2596), it seems to have a lesser influence on the incidence of type II EC (Murali R, Soslow R A, Weigelt B., The Lancet Oncology, 2014; 15(7):e268-78, doi: 10.1016/S1470-2045(13)70591-6). Most importantly, the majority of type II EC cases (80-90%) exhibit inactivating mutations in TP53 (the gene that encodes for the tumor suppressor protein, p53) (Hecht J L, et. al., Clinical Oncology. 2006; 24(29):4783-91, doi: 10.1200/JCO.2006.06.7173). In this regard, EC that exhibit loss of function (LOF) p53 can maintain the G2/M checkpoint in the cell cycle via the activation of compensatory anti-apoptotic P38/MK2 and the downstream components, which ultimately desensitize cancer cells against chemotherapies and induce chemo-resistance.

Due to the biological complexity and heterogeneity of tumors, and the emergence of various chemo-resistance mechanisms, combination chemotherapy (so-called chemotherapy cocktails) has therapeutic advantages over monotherapy (Hu Q, et. al., Advanced drug delivery reviews. 2016; 98:19-34, doi: 10.1016/j.addr.2015.10.022; and Bozic I, et. al., eLife. 2013; 2:e00747, doi: 10.7554/eLife.00747). These advantages may be attributed to the reversal of resistance, synergy, or additive effect, as these combinations also benefit from multiple mechanisms of all chemotherapeutics involved (Hu Q, et. al., Advanced drug delivery reviews. 2016; 98:19-34, doi: 10.1016/j.addr.2015.10.022; and Gerber H P, et. al., Cancer research. 2005; 65(3):671-80). For example, FOLFIRINOX is a chemotherapeutic cocktail, consists of folinic acid, 5-fluorouracil, irinotecan, and oxaliplatin, that is clinically used as a first-line treatment in advanced pancreatic ductal adenocarcinoma (PDAC) (Martin A M, et. al., Journal of Cancer. 2018; 9(11):1978-88, doi: 10.7150/jca.23716; and Ducreux M, et. al., Annals of oncology. 2015; 26 Suppl 5:v56-68, doi: 10.1093/annonc/mdv295). Another chemotherapeutic cocktail, nab-paclitaxel/gemcitabine, is also considered the first-line treatment for advanced PDAC (Wong H-L, et. al., Journal of Clinical Oncology. 2017; 35(4_suppl):468, doi: 10.1200/JCO.2017.35.4_suppl.468). Both combination therapy strategies showed improved overall survival against gemcitabine (FDA-approved drug for advanced PDAC), as FOLFIRINOX and nab-paclitaxel/gemcitabine achieved a median survival of 11.1 (Vaccaro V, et. al., The New England journal of medicine. 2011; 365(8):768-9; author reply 9, doi: 10.1056/NEJMc1107627) and 8.6 (Von Hoff D D, et. al., The New England journal of medicine. 2013; 369(18):1691-703, doi: 10.1056/NEJMoa1304369) months, respectively, compared to about 6.7-6.8 months with gemcitabine alone. Multiple mechanisms of these ingredients stand for improved efficacy. However, both strategies are associated with a high incidence of drug-related adverse events that may lead to treatment delay or cessation (Cho I R, et. al., World journal of gastrointestinal oncology. 2020; 12(2):182-94, doi: 10.4251/wjgo.v12.-i2.182) although nab-paclitaxel/gemcitabine may be associated with improved quality of life compared to FOLFIRINOX (Singh R R, O'Reilly E M. Drugs. 2020; 80(7):647-69, doi: 10.1007/s40265-020-01304-0). Similarly, a combination of paclitaxel (PTX) and carboplatin is widely used in endometrial cancer. As per FDA recommendation, the carboplatin dose is based on the Calvert formula as calculated by area under the plasma concentration-time curve (AUC) to avoid toxicity (Vergote I, et. al., Gynecologic oncology. 2015; 138(2):278-84, doi: 10.1016/j.ygyno.2015.05.042; and Vandenput I, et. al., International journal of gynecological cancer. 2012; 22(4):617-22, doi: 10.1097/IGC.0b013e31824a3385). PTX is potent chemotherapy but it has several side effects, among which, the most serious are those related to peripheral neuropathy and myelosuppression. These side effects are dose-related and dose-limiting (Scripture C D, et. al., Current Neuropharmacology. 2006; 4(2):165-72, doi: 10.2174/157015-906776359568; and Argyriou A A, et. al., Journal of pain and symptom management. 2006; 32(3):237-44, doi: 10.1016/j.jpainsymman.2006.03.013). Severe peripheral neuropathy occurs in up to 30% of patients receiving taxanes (especially PTX), and is sometimes associated with long-term irreversible functional disability (Lee J J, Swain S M. Journal of clinical oncology. 2006; 24(10):1633-42, doi: 10.1200/JCO.2005.04.0543; and Rivera E, Cianfrocca M. Cancer chemotherapy and pharmacology. 2015; 75(4):659-70, doi: 10.1007/s00280-014-2607-5).

Similarly, other approaches have been described in the literature to sensitize cancer cells towards PTX, abrogate their chemo-resistance machinery, and/or augment PTX efficacy. For instance, Zhang et al. reported the reversal of PTX chemo-resistance when a combination of PTX and the permeability-glycoprotein (P-gp) inhibitor (Tariquidar) was used against PTX-resistant ovarian cancer in vitro and in vivo (Zhang Y, et. al., Molecular cancer therapeutics. 2016; 15(10):2282-93, doi: 10.1158/1535-7163.MCT-15-0986). P-gp is one of the major cell membrane ATP binding cassette efflux transporters and constitutes one of the major mechanisms of Multi-Drug Resistance (MDR) (Sharom F J. Pharmacogenomics. 2008; 9(1):105-27, doi: 10.2217/14622416.9.1.105; and Leonard G D, et. al., The oncologist. 2003; 8(5):411-24, doi: 10.1634/theoncologist.8-5-411). PTX is a well-known substrate of P-gp (Allen J D, et. al., Cancer research. 2000; 60(20):5761-6; and Chico I, et. al., Journal of clinical oncology. 2001; 19(3):832-42, doi: 10.1200/JCO.2001.19.3.832), and the basal level of expression of the latter induces PTX resistance via reduction of its intracellular levels (Callies S, et. al., British journal of clinical pharmacology. 2003; 56(1):46-56, doi: 10.1046/j.1365-2125.2003.01826.x and Chico I, et. al., Journal of clinical oncology. 2001; 19(3):832-42, doi: 10.1200/JCO.2001-.19.3.832). Overexpression of P-gp for up to 100 times its basal levels can be found in about 50% of cancer patients (Wang F, Z et. al., Biomaterials. 2011; 32(35):9444-56, doi: 10.1016/j.biomaterials.2011.08.041), and reports confirm a correlation between p53-deficiency (like in LOF p53 cancer cells) and P-gp overexpression (Thottassery J V, et. al., Proceedings of the National Academy of Sciences of the United States of America. 1997; 94(20):11037-42, doi: 10.1073/pnas.94.20.11037; and Bush J A, Li G. Carcinogenesis. 2002; 23(10):1603-7, doi: 10.1093/carcin/23.10.1603), which further aggravates MDR. The use of P-gp inhibitors potentiates the efficacy of PTX by increasing its intracellular accumulation (De Vera A A, et. al., Cancer letters. 2019; 442:91-103, doi: 10.1016/j.canlet.2018.10.020) and improving its pharmacokinetics (PK) (Chico I, et. al., Journal of clinical oncology. 2001; 19(3):832-42, doi: 10.1200/JCO.2001.19.3.832; and Callies S, et. al., British journal of clinical pharmacology. 2003; 56(1):46-56, doi: 10.1046/j.1365-2125.2003.01826.x). On the other hand, drugs that stabilize microtubule assembly, by promoting tubulin polymerization, may synergize with PTX, a well-known microtubules stabilizer, and augment its anticancer activity, especially when both agents bind to different distinct tubulin binding sites (Rohena C C, Mooberry S L. Natural product reports. 2014; 31(3):335-55, doi: 10.1039/c3np70092e).

4N-Chlorophenyl carbamoylmethyl piperazin-lyl ciprofloxacin (CIP2b) was found to have moderate anticancer activity against various cancer cell lines (Mohammed H H H, et. al., Bioorganic & medicinal chemistry. 2016; 24(19):4636-46, doi: 10.1016/j.bm-c.2016.07.070). Topo I and II inhibitors like irinotecan (derivative of camptothecin, CAM) and etoposide (ETO), respectively, have shown benefits in gynecological cancers and are commonly used in clinical practice, either alone or in combination with PTX and other chemotherapeutics (Saif M W, Diasio R B. Clinical colorectal cancer. 2005; 5(1):27-36, doi: 10.3816/ccc.2005.n.014; Frumovitz M, et. al., Gynecologic oncology. 2017;1 44(1):46-50, doi: 10.1016/j.ygyno.2016.10.040; Aoki D, et. al., Japanese journal of clinical oncology. 2011; 41(3):320-7, doi: 10.1093/jjco/hyq192; and Holloway R W. Gynecologic oncology. 2003; 90(3 Pt 2):S28-33, doi: 10.1016/s0090-8258(03)00468-2). However, the side effects of such topoisomerase poisons comprise a roadblock that hinders the widespread application of these combinations (Pendleton M, Annals of the New York Academy of Sciences. 2014; 1310:98-110, doi: 10.1111/nyas.12358; and Oppegard L M, et. al., Investigational new drugs. 2019; 37(2):378-83, doi: 10.1007/s10637-018-0666-x). In contrast, ciprofloxacin (CIP), the parent compound of CIP2b, is known to inhibit the prokaryotic topoisomerase II, and is well-tolerated in patients.

Currently, there is a need for methods, agents, and compositions that are useful for treating cancer (e.g. endometrial cancer). For example, there is a need for methods, agents, and compositions that can achieve the same benefits of chemotherapeutic cocktails, allow the use of lower doses of PTX (but without compromising its efficacy), and consequently lower the rate of incidence of adverse events usually met with these multiple-agent combinations. There is also a need for methods, agents, and compositions that can improve the safety and efficacy of CIP2b, by increasing its tumor accumulation and minimizing generalized P-gp inhibition

### SUMMARY OF THE INVENTION

To maximize the efficacy of commonly used chemotherapeutics against endometrial carcinoma, the efficacy of a combination of a new ciprofloxacin derivative (CIP2b) with paclitaxel (PTX) was tested. The cytotoxicity of CIP2b with PTX was tested against loss of function p53 human endometrial cancer cells Hec50co that belong to type II adenocarcinoma, and the combination index was calculated to evaluate synergy. Cell cycle progress was also studied following treatment. To improve the intracellular uptake and in vivo performance, CIP2b was formulated in PLGA nanoparticles (NPs). Additionally, pharmacokinetics were tested in healthy mice and compared to a solubilized form of CIP2b in a surfactant-based vehicle. Several mechanisms of action of the combination were assessed, including P-gp inhibition, cellular accumulation enhancement, topoisomerase I and II inhibitory activity, tubulin polymerization rate, and anti-apoptotic mechanism. In vivo tumor accumulation of PTX±CIP2b as well as CIP2b NPs vs CIP2b solution were tested in Hec50co tumor-bearing mice. Finally, in vivo efficacy of the combination therapy was also assessed in athymic Nu/Nu mice bearing Hec50co tumors. Safety, tumor progress, and immunohistochemistry were also evaluated during the in vivo study.

Cytotoxicity assay revealed that there is a strong synergism between CIP2b and PTX against Hec50co. The effect was even potentiated when CIP2b NPs were used compared to the soluble counterpart. In vivo tumor accumulation of CIP2b was also improved when the NPs were used, as well as pharmacokinetics (area under the curve). Also, it was found that the mechanisms underlying this synergism can be attributed to the P-gp efflux inhibitory activity of CIP2b which significantly enhances the PTX accumulation inside cancer cells in vitro and in vivo. Furthermore, enzyme activity evaluation revealed that CIP2b combined with PTX strongly inhibits topoisomerase I and II, meanwhile, the addition of CIP2b potentiates the tubulin polymerization activity of PTX. These effects are associated with elevated caspase 3 and 9, but not 8, which indicate that the mitochondrial apoptosis pathway is involved in cell death. Finally, in vivo efficacy studies revealed that CIP2b NPs significantly improves the tumor accumulation of CIP2b following IV injection, and this led to enhanced antitumor activity when CIP2b NPs were co-injected with PTX. Histological examination revealed that this treatment strategy was safe, while immunohistochemical evaluation confirmed the previously obtained results, and hinted that an anti-vascular mechanism may still be involved.

Accordingly, the invention provides methods, agents, and compositions that are useful for treating cancer (e.g. endometrial cancer). The methods, agents, and compositions can achieve the same benefits of chemotherapeutic cocktails, allow the use of lower doses of taxanes (e.g. PTX), without compromising efficacy, and lower the rate of incidence of adverse events usually met with multiple-agent combinations.

In one aspect the present invention provides a method for treating cancer in an animal, comprising administering a taxane and a quinolone to the animal.

In another aspect, the invention provides a method comprising treating endometrial cancer in a human that has been diagnosed with endometrial cancer, by administering to the human a synergistic cancer treating amount of a combination of: a) paclitaxel or a pharmaceutically acceptable salt thereof, and 2) a compound of formula:

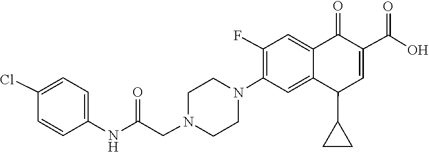 [SMILES: O=C(CN1CCN(c2cc3c(cc2F)C(=O)C(C(=O)O)=CC3C2CC2)CC1)Nc1ccc(Cl)cc1]

or a pharmaceutically acceptable salt thereof that is formulated with PLGA nanoparticles.

In another aspect, the invention provides a composition comprising a taxane, a quinolone, and a pharmaceutically acceptable carrier.

In another aspect, the invention provides a composition for treating endometrial cancer comprising, a synergistic endometrial cancer treating amount of a combination of: a) paclitaxel or a pharmaceutically acceptable salt thereof, 2) a compound of formula:

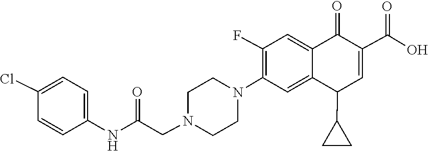 [SMILES: O=C(CN1CCN(c2cc3c(cc2F)C(=O)C(C(=O)O)=CC3C2CC2)CC1)Nc1ccc(Cl)cc1]

or a pharmaceutically acceptable salt thereof that is formulated with PLGA nanoparticles; and 3) a pharmaceutically acceptable carrier.

In another aspect, the invention provides a composition as described herein for use in medical therapy.

In another aspect, the invention provides a taxane for the prophylactic or therapeutic treatment of cancer, in combination with a quinolone.

In another aspect, the invention provides paclitaxel or a pharmaceutically acceptable salt thereof for the prophylactic or therapeutic treatment of endometrial cancer in combination with a compound of formula:

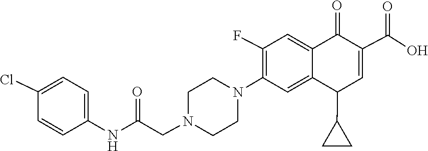 [SMILES: O=C(CN1CCN(c2cc3c(cc2F)C(=O)C(C(=O)O)=CC3C2CC2)CC1)Nc1ccc(Cl)cc1]

or a pharmaceutically acceptable salt thereof that is formulated with PLGA nanoparticles.

In another aspect, the invention provides the use of a taxane to prepare a medicament for the treatment of cancer in an animal, in combination with a quinolone.

In another aspect, the invention provides the use of paclitaxel or a pharmaceutically acceptable salt thereof to prepare a medicament for treating endometrial cancer in an animal, in combination with a compound of formula:

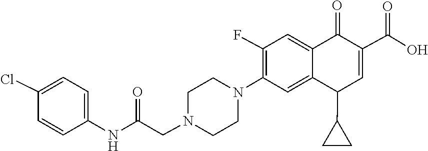 [SMILES: O=C(CN1CCN(c2cc3c(cc2F)C(=O)C(C(=O)O)=CC3C2CC2)CC1)Nc1ccc(Cl)cc1]

or a pharmaceutically acceptable salt thereof that is formulated with PLGA nanoparticles.

In another aspect, the invention provides a kit comprising: 1) packaging material, 2) a taxane, 3) a quinolone, and 4) instructions for administering the taxane and the quinolone to an animal to treat cancer.

In another aspect, the invention provides a kit comprising: 1) packaging material, 2) a pharmaceutical composition comprising paclitaxel or a pharmaceutically acceptable salt thereof; a compound of formula:

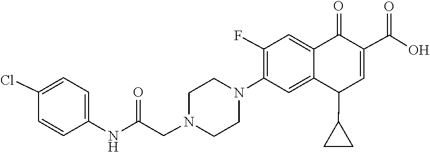 [SMILES: O=C(CN1CCN(c2cc3c(cc2F)C(=O)C(C(=O)O)=CC3C2CC2)CC1)Nc1ccc(Cl)cc1]

or a pharmaceutically acceptable salt thereof; and PLGA nanoparticles; and 3) instructions for the administration of the pharmaceutical composition for treating endometrial cancer in an animal.

The invention also provides processes disclosed herein for preparing pharmaceutical compositions of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1M: In vitro antitumor activity of CIP2b±PTX against Hec50co endometrial cells. FIG. 1A. Cytotoxicity assay following the treatment of Hec50co cells with different concentrations of PTX, and fixed concentrations of CIP2b (10, 25, and 50 μM) for 72 h. FIG. 1B. Cytotoxicity assay following the treatment of Hec50co cells with different concentrations of CIP2b, and fixed concentrations of PTX (1, 5, and 10 nM) for 72 h. FIG. 1C. Cytotoxicity assay following the treatment of Hec50co cells with different concentrations of PTX, and a fixed concentration (10 μM) of either CIP2b or CIP for 72 h. FIG. 1D. The synergy between CIP2b and PTX calculated using the Combination Index (CI) method as either a fixed concentration of PTX (5 nM) was added to CIP2b or a fixed concentration of CIP2b (10 μM) was added to PTX (CI values less than 1 indicate synergy). FIG. 1E. The effect of addition of 10 μM CIP2b to 1, 5, and 10 nM of PTX for 72 h. FIG. 1F. The effect of addition of 5 nM PTX to 1, 10, and 25 μM of CIP2b for 72 h. FIG. 1G. ICso values following the addition of 10, 25, and 50 μM of CIP2b to PTX. The letter ‘a’ indicates statistical significance (p<0.01) in comparison with PTX while ‘b’ indicates statistical significance (p<0.01) in comparison with PTX+10 μM CIP2b. FIG. 1H. IC₅₀ values following the addition of 1, 5, and 10 nM of PTX to CIP2b. The letter ‘a’ indicates statistical significance in comparison with CIP2b, ‘b’ indicates statistical significance (p<0.01) in comparison with CIP2b+1 nM PTX, and ‘c’ indicates statistical significance (p<0.01) in comparison with CIP2b+5 nM PTX. FIG. 1I. Cell cycle analysis of untreated cells, or cells treated with either 10 μM CIP2b, 5 nM PTX, 10 nM PTX, 5 nM PTX+10 μM CIP2b, or 10 nM PTX+10 μM CIP2b. FIG. 1J and FIG. 1J-1. Quantitative evaluation of cell cycle phases following the cell cycle analysis using ModFit LT (ver. 5.0.9, Verity Software House). FIG. 1K. Flow cytometric assay of the intracellular accumulation of PTX-OG (400 nM) in Hec50co cells in the presence or absence of CIP2b (4 or 40 μM). *** and **** indicate p<0.001 and 5 p<0.0001, respectively. FIG. 1L. Representative flow cytometric histograms of PTX intracellular accumulation experiment. FIG. 1M. Confocal microscopy images of PTX-OG with or without CIP2b in Hec50co cells.

FIG. 2A-FIG. 2O: Mechanistic studies to explore the mechanism(s) of action of the therapeutic combination of PTX and CIP2b. FIG. 2A, FIG. 2B, and FIG. 2C represent the levels of caspase 3, 8, and 9, respectively, in Hec50co after 24 h of treatment with PTX (5, and 10 nM), CIP2b (10, 25, and 50 μM), and their combinations. FIG. 2D, FIG. 2E, and FIG. 2F represent the levels of caspase 3, 8, and 9, respectively, in Hec50co cells after 72 h of treatment with PTX (5, and 10 nM), CIP2b (10, 25, and 50 μM), and their combinations. Statistical analysis was carried out by One-Way ANOVA followed by Tuckey's post-hoc test. *, **, *** and **** indicate p<0.05,p<0.01,p<0.001 and p<0.0001, respectively, while ‘ns’ indices not significant. FIG. 2G. Flow cytometric assay of the intracellular accumulation of PTX-OG (400 nM) in LLC-PK1-WT or LLC-PK1-MDR1 cells in the presence or absence of CIP2b (4 μM). FIG. 2I1. Cytotoxicity assay following the treatment of LLC-PK1-WT cells with different concentrations of PTX with or without 50 μM CIP2b for 72 h. FIG. 2I. Cytotoxicity assay following the treatment of LLC-PK1-MDR1 cells with different concentrations of PTX with or without 50 μM CIP2b for 72 h. FIG. 2J. Bright-field microscope images (utilizing a 10× objective lens (total magnification is 100×)) of LLC-PK1-MDR1 cells treated with PTX 10 nM or CIP2b 50 μM or a combination of both drugs where rounded cells represent cells undergoing apoptosis (under stress). Scale bar=400 FIG. 2K. Tubulin polymerization activity of PTX, CIP2b, and PTX+CIP2b. Tubulin and CaC12 were used as controls. FIG. 2L. Gel electrophoresis and FIG. 2M. bar graph showing the topoisomerase I inhibitory activity of CIP2b alone and in combination with PTX. FIG. 2N. Gel electrophoresis and FIG. 2O. bar graph showing the topoisomerase II inhibitory activity of CIP2b alone and in combination with PTX. In both topoisomerase assays, the controls used were PTX, CIP, PTX+CIP, camptothecin (CAM, in case of topoisomerase I), and etoposide (ETO, in case of topoisomerase II).

FIG. 3A-FIG. 3P: Molecular docking analysis of CIP2b and development of CIP2b PLGA NPs. FIG. 3A, FIG. 3B, FIG. 3C, FIG. 3D, and FIG. 3E display the molecular interaction of CIP2b with human P-gp, αβ-tubulin, topoisomerase I, topoisomerase IIα, topoisomerase IIβ, respectively. FIG. 3F. Proposed structure of the CIP2b-loaded PLGA NPs using TPGS as a surfactant. FIG. 3G. Scanning electron micrograph showing the shape and morphology of CIP2b NPs. Scale bar=100 nm. FIG. 3H. Properties of prepared CIP2b NPs. FIG. 3I. Cumulative in vitro release of CIP2b from NPs in release medium containing PBS and 0.1% w/v Tween-80 for 10 days. FIG. 3J. Cellular uptake of either CIP2b NPs or soluble CIP2b by Hec50co cells. FIG. 3K. Cytotoxicity assay following the treatment of Hec50co cells with different concentrations of PTX, and fixed concentrations of either CIP2b NPs (equivalent to 0.1 or 0.5 μM of CIP2b) or blank NPs (equivalent to the amount of CIP2b NPs used) for 72 h. FIG. 3L. The effect of addition of CIP2b NPs (equivalent to 0.1 or 0.5 μM CIP2b) or blank NPs (equivalent to the amount of CIP2b NPs used) to 0.1 nM PTX for 72 h. The letter ‘a’ indicates statistical significance (p<0.01) in comparison with PTX, ‘b’ indicates statistical significance (p<0.01) in comparison with PTX+0.1 μM blank NPs, and ‘c’ indicates statistical significance (p<0.01) in comparison with PTX+0.5 μM blank NPs. FIG. 3M. Cytotoxicity assay following the treatment of Hec50co cells with different concentrations of CIP2b NPs (equivalent to 0.01, 0.1, and 0.5 μM CIP2b) or blank NPs (equivalent to the amount of CIP2b NPs used) for 72 h. The letters ‘a’ and ‘b’ indicate statistical significance (p<0.001 and p<0.0001, respectively) in comparison with the corresponding concentrations of blank NPs. FIG. 3N. Cell cycle analysis of Hec50co cells treated with either 10 nM PTX+CIP2b NPs equivalent to 0.5 μM of CIP2b, or 10 nM PTX+blank NPs equivalent to the amount of CIP2b NPs used. FIG. 3O. Representative flow cytometric histograms of PTX intracellular accumulation experiment. FIG. 3P. Flow cytometric assay of the intracellular accumulation of PTX-OG (400 nM) in Hec50co cells in the presence or absence of either CIP2b NPs (equivalent to 4 μM CIP2b) or blank NPs (equivalent to the amount of CIP2b NPs used). Statistical analysis was carried out by One-Way ANOVA followed by Tuckey's post-hoc test. *** and **** indicate p<0.001 and p<0.0001, respectively.

FIG. 4A-FIG. 4P: In vivo antitumor efficacy of PTX+CIP2b. FIG. 4A. Graphical representation of the in vivo tumor cells uptake study of PTX-OG in the presence or absence of CIP2b. Briefly, Hec50co tumor cell uptake PTX-OG following its peri-tumoral subcutaneous injection (SC) with or without CIP2b. Tumors were collected, sliced into small pieces, incubated in cell dissociation buffer, and processed into a single-cell suspension. Cells were washed thoroughly and finally analyzed by flow cytometry for PTX-OG fluorescence. FIG. 4B. Histograms following flow cytometric analysis of the tumor cells (i.e., single-cell suspension). FIG. 4C. Bar graphs representing the PTX-OG fluorescence mean of the 3 study groups. Statistical analysis was carried out by One-Way ANOVA followed by Tuckey's post-hoc test. * and ** indicate p<0.05 and p<0.01, respectively. FIG. 4D. Hec50co tumor (human endometrial cancer xenografts) progress in athymic Nu/Nu mice following treatment with PBS, PTX, or PTX+CIP2b solution by SC injection near the tumor site (i.e., peritumorally). Treatment started on day 18 of tumor challenge (1×10⁶ cells/mouse). On day 53, mice were euthanized, and their tumors were collected and weighed. * indicates p<0.05. FIG. 4E. Body weight monitoring of the mice following their SC treatment with PBS, PTX or PTX+CIP2b solution. FIG. 4F. Photographic image of tumors collected from mice treated with PBS, PTX or PTX+CIP2b solution. FIG. 4G. Average tumor weights following mice euthanasia and tumor collection from mice treated with PBS, PTX, or PTX+CIP2b solution. Statistical analysis was carried out by a 2-tailed Student's T-test. * indicates p<0.05. FIG. 4H. Hec50co tumor progress in athymic Nu/Nu mice following treatment with PBS, PTX+Blank NPs, PTX+CIP2b solution, or PTX+CIP2b NPs by intravenous injection (IV). Treatment started on day 18 of tumor challenge (1×10⁶ cells/mouse). On the 53^{rd} day, mice were euthanized, and their tumors were collected and weighed. * indicates p<0.05. FIG. 4I. Body weight monitoring of the mice following their IV treatment with PBS, PTX+Blank NPs, PTX+CIP2b solution, or PTX+CIP2b NPs. FIG. 4J. Photographic image of tumors collected from mice treated with PBS, PTX+Blank NPs, PTX+CIP2b solution, or PTX+CIP2b NPs. FIG. 4K. Average tumor weights following mice euthanasia and tumor collection from mice treated with PBS, PTX+Blank NPs, PTX+CIP2b solution, or PTX+CIP2b NPs. Statistical analysis was carried out by 2-tailed Student's T-test. * indicates p<0.05. FIG. 4L. Immunohistochemistry analysis of tumors collected from representative mice treated by IV injection of PBS, PTX+Blank NPs, PTX+CIP2b solution, or PTX+CIP2b NPs after staining with H&E, or antibodies against CD31, caspase 3, ki-67, or β-tubulin. Scale bar=1 inch. FIG. 4M. Histological evaluation of major vital organs following the IV administration of PBS, PTX+Blank NPs, PTX+CIP2b solution, or PTX+CIP2b NPs after H&E staining of samples from the organs collected from representative mice from each group. Scale bar=1 inch. FIG. 4N. Plasma concentrations-time curves following the IV injection of CIP2b NPs or CIP2b solution (25 μM each) in female Balb-c mice. FIG. 4O. Pharmacokinetics parameters following the non-compartmental analysis of the plasma concentrations data. FIG. 4P. Intra-tumoral accumulation of CIP2b following the IV injection of either CIP2b solution or CIP2b NPs in Hec50co tumor-bearing athymic Nu/Nu mice. Statistical analysis was carried out by a 2-tailed Student's T-test. * represents p<0.05 compared to CIP2b solution.

FIG. 5: Shows the pKa for the compound CIP2b determined using the following solubility equation: log S=log Sₒ+log(10^{-pK}^{ₐ}^{+pH}+1)=(log Sₒ−pKₐ)+pH, where intercept=(log Sₒ−pKₐ) and slope=1 [1]. From the solubility (log S) vs pH profile graph based on the above equation, the intrinsic solubility was determined, log Sₒ, is −0.26 or 0.5 μg/mL.

FIG. 6A-FIG. 6D: Biodistribution of CIP2b following the IV injection of 75 μg of CIP2b solution (top left), and CIP2b NPs (top right), and 25 μg CIP2b solution (bottom left) and CIP2b NPs (bottom right) in female Balb-c mice. Values are means±SD, n=3.

FIG. 7A-FIG. 7C-1: Cytotoxicity of chemotherapeutic agents and their combinations in different cancer cell lines. FIG. 7A. cytotoxicity of 5-FU, CIP2b, PTX, and their combinations in murine colon cancer cells. The top figure shows that CIP2b does not enhance the effect of 5-FU, while the middle and bottom figures show that there is strong synergism between CIP2b and PTX. FIG. 7B. cytotoxicity of CIP2b and PTX against MDA-MB-468. CIP2b (10 p,M) does not improve the efficacy of PTX against this cell line. FIG. 7C and Figure C-1. Anticancer activity of CIP2b either alone or after adding 50 and 75 nM of AZD7762 or 150 and 250 nM of MK1775 (adavosertib) to CIP2b. No synergism can be noticed. Data are means±SD, n=4.

### DETAILED DESCRIPTION OF THE INVENTION

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of the extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiologically acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Cancer

Cancers that can be treated using this combination may include any type of cancer that exhibits resistance against taxanes, especially due to MDR1 gene overexpression (P-gp), and other cancers that respond to Topoisomerase I and/or II inhibition, or those that respond to tubulin polymerization stabilizers. In one embodiment, the cancer is selected from endometrial cancer, pancreatic cancer, colon cancer, and glioblastoma. In one embodiment, the cancer is endometrial cancer.

### Taxanes

Taxanes are chemotherapeutic agents derived from the yew tree species (e.g. Taxus brevifolia and Taxus baccata). They are considered the first line of treatment in many cancers, either alone or in combinations, like breast cancer, pancreatic cancer and ovarian cancer. In one embodiment, the taxane is paclitaxel or docetaxel, or a pharmaceutically acceptable salt thereof. In one embodiment, the taxane is paclitaxel or a pharmaceutically acceptable salt thereof.

### Quinolones

Fluoroquinolones are antibacterial agents that inhibit bacterial DNA gyrase and topoisomerase IV, and they include ciprofloxacin, gatifloxacin, and levofloxacin. Some chemically modified fluoroquinolones induce cancer cell death by inhibiting topoisomerase II, especially when the modification takes place at the 4N location (Mohammed, H. H. H., et al., Bioorg Med Chem, 2016. 24(19): p. 4636-4646). Other 1N derivatives are also known to possess anticancer activity via inhibition of topoisomerase I (Oppegard, L. M., et al., Invest New Drugs, 2019. 37(2): p. 378-383). In one embodiment, the quinolone is a fluoroquinolone. In one embodiment, the quinolone has topoisomerase inhibitory activity. In one embodiment, the quinolone has both topoisomerase-1 inhibitory activity and topoisomerase-2 inhibitory activity. In one embodiment, the quinolone has P-gp efflux pump inhibitory activity. In one embodiment, the quinolone has microtubule stabilizing activity. In one embodiment, the quinolone is:

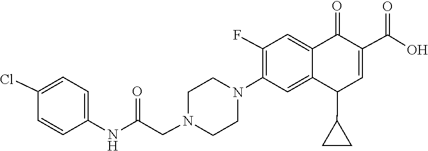 [SMILES: O=C(CN1CCN(c2cc3c(cc2F)C(=O)C(C(=O)O)=CC3C2CC2)CC1)Nc1ccc(Cl)cc1]

or a pharmaceutically acceptable salt thereof.

### Nanoparticles

The compositions having one or more CIP2B modulators may include or may be formed from biodegradable polymeric molecules which may include, but are not limited to polylactic acid (PLA), polyglycolic acid (PGA), copolymers of PLA and PGA (e.g., polyactic-co-glycolic acid (PLGA)), poly-€-caprolactone (PCL), polyethylene glycol (PEG), poly(3-hydroxybutyrate), poly(pdioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (pcarboxypheonoxy) methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], polyhydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 5 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425.

Nanoparticles may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425. Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, nanoparticles have a mean effective diameter of less than 1 micron, e.g., the nanoparticles have a mean effective diameter of between about 25 nm and about 500 nm, e.g., between about 50 nm and about 250 nm, about 100 nm to about 150 nm, about 150 nm to about 175 nm, about 150 nm to about 200nm, about 400 nm to about 450 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight 5 Mass Spectroscopy (ATFMS), and Aerosol Particle Mass Analyzer (APM).

Nanoparticles may have a zeta-potential that facilitates uptake by a target cell. Typically, the nanoparticles have a zeta-potential greater than 0. In some embodiments, the nanoparticles have a zeta-potential between about 5 mV to about 45 mV, between about 15 mV to about 35 mV, or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a composition comprising one or more CIP2B modulators comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic 20 liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; chitosan, PEI-polyethylene glycol, PEI-mannose-dextrose, DOTAPcholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (Dglucarate (D), meso-galactarate (G), D-mannarate (M), and L-tartarate (T)) with a 30 series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles 5 coated with PAMAM, or any combination thereof. The disclosed polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammoniumpropane (DOTAP); N-[1-(2,3-dimyristloxy) propyl]; N,N-dimethyl-N-(2-hydroxyethyl) ammonium bromide (DMRIE), 3-β-[V-(N,N-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium 20 group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of NN-dioleyl-N,N-dimethylammonium chloride (DODAC).

The structures of polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central 5 core molecule, from which several highly branched arms ‘grow’ to form a tree-like structure with a manner of symmetry or asymmetry. Examples of dendrimers include polyamidoamine (PAMAM) and polypropylenimine (PPI) dendrimers. DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used. In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres. In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N30 isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination 5 of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L15 lactide-co-glycolide, polyglycolide and poly(D,L-lactide), pol(D,L-lactidecoglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(Ecaprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], polyhydroxybutyric acid, or S-caproic acid, polylactide-co-glycolide, polylactic acid, polyethylene glycol, cellulose, oxidized cellulose, alginate, 5 gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acidpara-dioxanone-polyethylene glycol block copolymer, polypropylene, poly(ethyleneterephthalate), poly(tetrafluoroethylene), poly-epsilon-caprolactone, or crosslinked chitosan hydrogels.

In one embodiment, the compositions comprising the CIP2B modulators may comprise microparticles or microspheres (size ranges from 300 nm to less than 1mm), which can be made from or comprise one or more of the above-mentioned materials.

In one embodiment, the compositions comprising the CIP2B modulators may be fabricated in the form of pellets or implants for subcutaneous insertion, which can be made from or may comprise one or more of the above-mentioned polymers.

In one embodiment, the quinolone is formulated with nanoparticles.

In one embodiment, the nanoparticles are PLGA nanoparticles.

In one embodiment, the nanoparticles have a size of from about 25 nm to about 500 nm.

The invention will now be illustrated by the following non-limiting Examples.

### EXAMPLES

### Example 1. Biological Evaluations of Paclitaxel and CIP2B

### Materials and Methods

Compounds and reagents. Amicon® Ultra-15 Centrifugal Filter Unit, Ultracel-100 regenerated cellulose membrane (molecular weight cutoff 100 kDa) (Cat. No. UFC9100), CAM (Cat. No. 208925), TPGS (Cat. No. 57668), dimethyl sulfoxide (DMSO, Cat. No. D2438), DNase I from bovine pancreas (Cat. No. DN25), ETO (Cat. No. 341205), 7-hydroxyflavone (Cat. No. H4530), and Float-A-Lyzer G2 Dialysis Devices, 8-10 kD (Cat. No. G235025) were purchased from Sigma Aldrich (St. Louis, MO). Ciprofloxacin (Cat. No. 449620050), Collagenase Type IV (Cat. No. 17104019), NP-40 Surfact-Amps™ 10% Detergent Solution (Cat. No. 28324), Oregon Green™ 488 PTX (PTX-OG, Cat. No. P22310), propidium iodide (PI) 1 mg/mL (Cat. No. P3566), RNase A (DNase and protease-free) 10 mg/mL (Cat. No. EN0531), Tween-80 (Cat. No. BP338-500), and UltraPure™ Ethidium Bromide 10 mg/mL (Cat. No. 15585011) were purchased from ThermoFisher Scientific (Waltham, MA). CellTiter 96 ® AQueous One Solution Cell Proliferation Assay (MTS) (Cat. No. G3581) was purchased from Promega (Madison, WI). Disruption beads, 2.3 mm Zirconia/Silica Beads (Cat. No. 11079125z) were purchased from BioSpec (Bartlesville, OK). Paclitaxel powder (Cat. No. P-9600) was purchased from LC Laboratories (Woburn, MA) while Paclitaxel concentrate (6 mg/mL, Cat. No. 70860-200-05) for IV infusion was purchased from Athenex, Inc. (Buffalo, NY). Poly(D,L-lactide-co-glycolide) 50:50 (PLGA, RESOMER® RG 502 H) was purchased from Evonik Industries AG (Darmstadt, Germany). All other reagents, buffers, and solvents were at least of analytical grade and were used as received without further purification.

Cells and cell culture materials. Hec50co cells, a subline of Hec50 cells, were generously provided by Professor Erlio Gurpide (New York University). Hec50co cells were cultured in Dulbecco's modified Eagle's medium 1× (DMEM, Cat. No. 11965-092) supplemented with 1% Penicillin/Streptomycin (Pen/Strep, 100 U/mL, Cat. No. 15140122) and 10% fetal bovine serum (FBS, Cat. No. 511150) (ThermoFisher Scientific). Lewis-lung cancer porcine kidney cell lines LLC-PK1-WT (wild-type) and LLC-PK1-MDR1 (overexpressing P-gp, Multidrug Resistance Mutation 1 gene) were generously provided by Professor John Markowitz (University of Florida) and were cultured in DMEM supplemented with 1% Pen/Strep, 10% FBS, and 10 mg/mL sodium pyruvate (Cat. No. 11360-070, ThermoFisher Scientific). For trypsinization, 0.25% Trypsin-EDTA 1× (Cat. No. 25200-056, ThermoFisher Scientific) was used. All the cells were incubated and maintained in a Galaxy ® 170S CO₂ humidified cell culture incubator (Eppendorf, Germany) set at 37° C. under a 5% CO₂ flow.

Synthesis of CIP2b. CIP2b was synthesized and purified as previously described (Mohammed H H H, et. al., Bioorganic & medicinal chemistry. 2016; 24(19):4636-46, doi: 10.1016/j.bmc.2016.07.070). Physicochemical characterization was carried out to identify CIP2b properties like FTIR spectrum, solubility, pH-solubility profile, pKa, and partition coefficient.

### In Vitro Studies on Hec50co Cells.

Cytotoxicity assay. Hec50co cells were seeded at 1500 cells per well in 96-well plates for 24 h in a complete medium. Later, the medium was removed and PTX, CIP2b, or CIP (all dissolved in DMSO and diluted in complete medium) were added to cells as either monotherapies or in combinations, and the plates were incubated for 72 hours. Then, the complete medium was removed and MTS reagent in the complete medium was added to wells, and the plates were incubated for 1-4 hours according to the manufacturer's protocol. Finally, the absorbance was recorded at 490 nm using a Spectra Max plus 384 Microplate Spectrophotometer (Molecular Devices, Sunnyvale, CA). Percentage cell viability was expressed as the average absorbance of the test group relative to that of the control group (untreated cells). The concentration that inhibits proliferation of 50% of cells compared to control (IC₅₀) was calculated using GraphPad Prism (La Jolla, CA). Synergism was detected following the Chou-Talalay method (CI<1 indicates synergism) (Chou T C. Cancer research. 2010; 70(2):440-6, doi: 10.1158/0008-5472.CAN-09-1947) using CompuSyn software (Ver. 1.0).

Cell Cycle assay. Hec50co cells were seeded at 250×10⁵ cells per well in 6-well plates for 48 h in complete medium. The medium was then removed and replaced with PTX (1, 5, and 10 nM), CIP2b (10 μM), combinations of PTX with CIP2b, or with a fresh complete medium. Cells were incubated with the treatments for 24 h, then the medium was removed, and the cells were washed twice with Dulbecco's Phosphate-Buffered Saline (DPBS 1×, ThermoFisher Scientific), trypsinized (0.4 mL/well), and collected by centrifugation (230 xg, 5 min). Cells were then fixed using 70% ethanol stored at 4° C. for 30 min, then incubated with 100 μg/mL RNase A in 2% v/v NP-40 Surfact-Amps® solution (0.5 mL/sample) for 30 min at 37° C. Then, PI (50 μg/mL, 0.5 mL/sample) was added prior to analysis by flow cytometry (FACScan, BD Biosciences) using CellQuest software (Ver. 3.3). The data were further analyzed by ModFit LT (Ver. 5.0) to calculate the percent of cells in each phase of the cell cycle (i.e., G0/G1, S, G2/M).

Intracellular accumulation of PTX using flow cytometer. To evaluate PTX cellular accumulation in the presence or absence of CIP2b, Hec50co cells were seeded at a density of 200,000 cells per well in 6-well plates in complete medium and incubated for 24 h. Afterward, the medium was removed, and a DMSO-based solution (0.1 mg/mL) of fluorescently labeled PTX (PTX-OG) was added at 400 nM per well, and the volume was completed to 2 mL with the medium. Also, CIP2b (in DMSO) was added at 4 or 40 μM, either alone or in combination with PTX-OG. After 2 h, the medium was removed, the cells were washed thoroughly with DPBS, trypsinized, and collected by centrifugation (230 xg, 5 min). Finally, cells were analyzed by flow cytometry (FACScan).

Intracellular accumulation of PTX using confocal microscopy. Intracellular accumulation of PTX co-delivered with CIP2b was also studied qualitatively in Hec50co cells using confocal microscopy. In this experiment, Hec50co cells were seeded in 4-well chambered cover glass with non-removable wells (Cat. No. 155383, ThermoFisher Scientific) at a density of 3×10⁴ cells in a 0.8 mL complete medium per well and incubated for 24 hours. Next, the medium was carefully aspirated, and cells were incubated with different treatments (400 nM PTX-OG, or 40 μM CIP2b, or a combination of both compounds) for 2 hours. Then, the medium was removed, and this was followed by washing twice with 1× live cell imaging solution (Cat. No. A14291DJ, ThermoFisher Scientific). Finally, samples were visualized using Leica TCS SP8 STED confocal laser scanning microscope (Leica Microsystems Inc., Buffalo Grove, IL), and the confocal images were processed using the Image)-based Fiji software.

### Mechanistic Studies

Apoptosis and caspase activity. The expression of caspase 3, 8, and 9 was measured using Multiplex Activity Assay Fluorometric Kit (Cat. No. ab219915, Abcam). Briefly, Hec50co cells were plated in 96-well plates at a density of 3000 per well in 100 μL of the complete medium and incubated for 24 hours. After incubation, the medium was removed, and different concentrations either in single or combination of treatments were added (PTX 5 and 10 nM; CIP2b 10, 25, and 50 μM). Cells with treatments were incubated for 24 or 72 hours. Then, the caspase kit reagents were added to the cells and incubated at room temperature for 60 minutes. Fluorescence intensity (at excitation/emission values of 535/620 nm, 490/525 nm, and 370/450 nm for caspase 3, 8, and 9, respectively) was then measured using a SpectraMax M5 microplate reader (Molecular Devices).

P-gp efflux transporter. Overexpression of the MDR1 gene, reflected in the abundance of the P-gp efflux pump, is one of the major reasons for PTX chemoresistance in cancer [4]. In this study, LLC-PK1-WT and LLC-PK1-MDR1 cell lines were used. MTS assay and the PTX intracellular accumulation experiment were performed as previously described above.

Tubulin polymerization assay. Microtubules are one of the major cytoskeleton components in the cells, and they play an essential role in the spindle formation generated by α and β tubulin polymerization. Agents targeting microtubules function through two different mechanisms. To elaborate, several compounds like taxanes (e.g., PTX) promote the assembly of microtubules and are known to stabilize the microtubes whereas other compounds such as vinca alkaloid and colchicine are known to interrupt the spindle formation via inhibiting the tubulin polymerization; thereby, destabilize the microtubules. The result of both scenarios is metaphase arrest which induces apoptosis. To test the tubulin activity, a fluorescence-based tubulin polymerization assay (Cat. No. BK011P, Cytoskeleton Inc., Denver, CO) was performed according to the manufacturer's instructions. Briefly, stock solutions of PTX and CIP2b (in DMSO) were diluted with UltraPure™ water to make a final concentration of 3 μM. In addition, a 75 μM CaCl₂ aqueous solution was used as a control in this experiment (to inhibit tubulin polymerization). Before starting the experiment, the 96-well plate and fluorimeter (SpectraMax M5 Microplate Reader) were pre-warmed to 37° C. The fluorimeter was set up for kinetic measurements every minute for 1 hour at excitation 360 nm and emission 450 nm. Samples and tubulin were added to a 96-well plate, and the plate was inserted into the plate reader. Kinetic measurements started immediately, and data were collected and analyzed.

Topoisomerase I inhibition assay. A plasmid-based Topo I assay (Cat. No. TG1015-1A, TopoGEN Inc., Buena Vista, CO) was performed according to the manufacturer's instructions. Briefly, 1 μL of the test compounds (PTX, CIP, CIP2b, or CAM) at a concentration of 2 mM, was mixed with 2 μL of the reaction buffer (10× tris-glycine-SDS), and deionized water was added to make 18 μL solution. This was followed by adding 1 μL of supercoiled DNA (SC DNA) and 1 μL of human Topo I (10 U/μL) (drug final concentration was 100 μM). Also, SC DNA±Topo I was used as a control (i.e., not containing drugs). In addition, relaxed DNA was added as another control in this assay. Samples were incubated on a heating block (Isotemp 125 D Digital Heat Block, ThermoFisher Scientific) set at 37° C. for 30 minutes. Then, the reaction was stopped by adding a 5× stop buffer. Samples were then loaded directly into the wells of a 1% agarose gel (Research Products International, Mount Prospect, IL). The gel was run at 150 V until the dye front had traveled approximately 5 cm (run time was approximately 2 hours). The gel was then stained with 1 μg/mL ethidium bromide in deionized water for 15 minutes, and this was followed by de-staining (washing) with deionized water for 30 min before imaging. The image was then taken using an Invitrogen iBright CL 1500 Imaging System (ThermoFisher Scientific). The intensity of the final bands in the gel was quantified using ImageJ, and the results are expressed as the percentage of Topo I inhibition.

Topoisomerase II inhibition assay. A plasmid-based Topo II assay (Cat. No. TG1001-1A, TopoGEN Inc., Buena Vista, CO) was performed according to the manufacturer's instructions. In this experiment, 1 μL of tested compounds (PTX, CIP, CIP2b, ETO) at a concentration of 2 mM was mixed with 4 μL of the reaction buffer (mixture of buffer A (0.5 M Tris-HCl (pH 8), 1.50 M NaCl, 100 mM MgCl₂, 5 mM Dithiothreitol, 300 μg BSA/mL) and buffer B (10× ATP Buffer), and deionized water was added to make 18 μL solution. This was followed by adding 1 μL of catenated DNA and 1 μL of human Topo IIα (8 U/μL) (drug final concentration was 100 μM). Additionally, decatenated DNA (De-CT DNA) and catenated DNA (CT DNA)±Topo II were used as a control (i.e., not containing drugs). Moreover, relaxed DNA was added as another control in this assay. Similar to Topo I assay, samples were then incubated on a heating block set at 37° C. for 30 minutes. Then, the reaction was stopped by the addition of a 5× stop buffer. Samples were then loaded directly into the wells of a 1% agarose gel containing 1 μg/mL ethidium bromide. The gel was run at 150 V until the dye front had traveled approximately 5 cm (run time was approximately 2 h). The gel was then washed with deionized water for 30 minutes before imaging. The image was taken using an Invitrogen iBright CL1500 Imaging System. The intensity of the final bands in the gel was quantified using ImageJ, and the results are expressed as the percentage of Topo II inhibition.

Molecular docking simulation. The 3D crystallographic structures of targets (human Topo I (PDB ID: 1T8I), Topo II alpha (PDB ID: SGWK), Topo II beta (PDB ID: 3QX3), P-gp (PDB ID: 6QEX), and αβ-tubulin (PDB ID: 1JFF) were retrieved from the RCSB Protein Data Bank (PDB) database. Water molecules, bound antibodies, unnecessary ions, and other hetero atoms were removed from the structures using BIOVIA Discovery Studio Visualizer v20.1.0.19295 (Accelrys) program. Also, the 2D structure of the ligand CIP2b was drawn in ChemDraw (version; 20.1.0.110) and converted to a 3D structure by Discovery Studio Visualizer. Energy minimization of the ligand was conducted by the Merck molecular force field 94 (MMFF94), and Gasteiger was the charge calculation method (Naguib Y W, et. al., Biomaterials. 2021; 275:120842, doi: 10.1016/j.biomaterials.2021.120842). Rigid docking was chosen by converting all rotatable bonds of the ligand to non-rotatable to minimize standard errors coming from the rotatable bonds during the docking simulation (Trott O, et. al., J Comput Chem. 2010; 31(2):455-61, doi: 10.1002/jcc.21334). The binding site was analyzed in Discovery Studio Visualizer for targeted site-specific docking and grid box parameters were obtained covering the target binding site. Missing residues, polar hydrogens, Kollman charges, and Gasteiger charges were added to the protein structures using AutoDock Tools (ADT), a free graphical user interface of MGL software packages v1.5.7rc1 (Morris G M, et. al., J Comput Chem. 2009; 30(16):2785-91, doi: 10.1002/jcc.21256). Molecular docking simulation was conducted using AutoDock Vina (version 1.1.2) (Trott O, et. al., J Comput Chem. 2010; 31(2):455-61, doi: 10.1002/jcc.21334). Lamarckian Genetic Algorithm (LGA) was used to find the greatest conformational space for the ligand with a population of 150 individuals. The maximum number of generations and evaluations was set at 27,000 and 2,500,000, respectively (Naguib Y W, et. al., Biomaterials. 2021; 275:120842, doi: 10.1016/j.biomaterials.2021.120842). The best docked pose was selected based on the lowest binding energy. Simulation results were analyzed by Discovery Studio Visualizer and PyMOL version 2.3.

### Fabrication and Characterization of the CIP2b-Loaded PLGA Nanoparticles

Preparation. CIP2b-loaded PLGA nanoparticles (CIP2b NPs) were prepared using the nanoprecipitation technique. Briefly, 1 mg of CIP2b and 3 mg of PLGA were dissolved in 1.5 mL of a mixture of acetone and methanol (9:1). This solution was added dropwise into an 0.1% w/v TPGS aqueous solution in UltraPureTM water (DNase/RNase-free distilled water) stirred at 400 rpm using a syringe and needle (G26, ½ inch) with the tip of the needle submerged right below the surface of the aqueous solution. After CIP2b NPs were formed, the colloidal suspension was transferred to a round bottom flask and the organic solvents were removed under vacuum (50 mbar) using a rotary evaporator for 2 h (RotaVap R300, Buchi Labortechnik AG, Switzerland). The surfactant was removed using Amicon® ultra-15 centrifugal filter units following centrifugation and washing with UltraPure™ water 3-4 times (500 xg, 30 minutes/each). CIP2b NPs were finally collected and used directly. Blank (drug-free) NPs were prepared using the same method but without adding the drug.

Particle size and zeta potential measurements. CIP2b NPs or blank NPs were diluted 100-fold with NanoPure™ water, and the particle size and zeta potential were determined using Malvern Zetasizer Nano ZS (Malvern Panalytical, Westborough, MA).

Particle shape and morphology. A Hitachi S-4800 scanning electron microscope (SEM, Hitachi High-Technologies, Ontario, Canada) was used to examine the shape and surface morphology of the prepared CIP2b NPs. First, a droplet of the diluted NP suspension was placed onto a silicon wafer on an aluminum stub, and it was left to air-dry for 24 hours. Then, it was sputter-coated with iridium using an EMS150T-ES sputter coater (Quorum Technologies, Lewes, UK). Finally, the sample was scanned using the SEM operated at 5kV accelerating voltage.

CIP2b content measurement. Briefly, 10 μL of the NP suspension was dissolved in 90 μL of acetonitrile, then 0.9 mL of methanol was added, and the solution was sonicated for 30 seconds. Finally, 1 mL of NanoPureTM water was added. The solution was centrifuged (16,000 xg, 5 minutes) and injected directly into the HPLC for content measurement. An Agilent HPLC system was used for the CIP2b assay (Agilent Infinity 1100, Santa Clara, CA). A Waters Symmetry RP-C18 column was used for the assay (5 μm pore size, 4.6 mm×150 mm, Milford, MA). To quantify the CIP2b, an isocratic elution was used, and the mobile phase consisted of methanol:water 50:50. The flow rate was 1 mL/minute at room temperature and the injection volume was set to be 50 μL. The detection wavelength was set to 275 nm. Drug content (μg CIP2b/mL NP suspension) was calculated using Equation 1.

$$Drug content = \frac{Amount of CIP 2 b in the final formulation ( μg )}{The total volume of the CIP 2 b NP suspension ( mL )} \qquad Equation 1$$

In vitro release kinetics. CIP2b NPs (containing 120 μg of CIP2b) were diluted in NanoPure™ water to a total volume of 1 mL and added to pre-rinsed Float-A-Lyzer G2 tubes. The Float-A-Lyzer tubes were placed in 12 mL of the release medium (DPBS 1×, pH 7.4 with 0.4% w/v Tween-80) inside a 50-mL Falcon tube (n=3). The tubes were incubated at 37 ° C. and shaken at 300 rpm in an orbital incubator-shaker (New Brunswick Scientific, Edison, NJ). At pre-determined time points, the whole volume inside the Falcon tubes was removed and replaced with a fresh medium. CIP2b content in the samples was analyzed using HPLC as described above.

In vitro activity of CIP2b NPs. The cytotoxic activity of the CIP2b NPs and blank NPs was tested against Hec50co cells following the same procedure described above. The CIP2b equivalent concentration of CIP2b NPs was added instead of the CIP2b solution. An equivalent quantity of blank NPs was also tested to account for the effect of the PLGA NPs alone without CIP2b. Similarly, cell cycle analysis was tested following incubation of Hec50 cells with CIP2b NPs instead of CIP2b solution, as described above. Also, the intracellular accumulation of PTX co-administered with CIP2b NPs was investigated. CIP2b NPs were added at 4 or 40 μM, either alone or in combination with PTX-OG (400 nM). An equivalent quantity of blank NPs was also added, either alone or with PTX-OG. Samples were then analyzed using a flow cytometer, as described above.

### Animal Studies

Research animals. Female Balb-c mice (6-8 weeks, Jackson Labs, Sacramento, CA) or female athymic Nu/Nu mice (6-8 weeks, Charles River, Wilmington, MA) were used in animal studies. Mice were kept under controlled temperature (23±2° C.) at the University of Iowa animal care facility. Food was provided ad libitum, and mice were exposed to 12 hours of light/dark cycles. All animal experiments performed were approved by the University of Iowa Institutional Animal Care and Use Committee (IACUC). When anesthesia was needed, mice were injected IP with the ketamine-xylazine mixture (87.5 mg/kg ketamine and 12.5 mg/kg xylazine) prior to performing the experiment.

Effect of drug combination on the in vivo tumor accumulation of PTX In this study, athymic Nu/Nu mice were challenged with 1×10⁶ of Hec50co tumor cells per mouse on day zero of the experiment. When the tumor grew to a palpable size range of 100-150 mm³, as measured by Equation 2, treatments were administered SC at the tumor site (peritumoral). Treatment groups (n=3 per group) included: (i) DPBS treated mice, 0.1 mg/kg PTX-OG, and 0.1 mg/kg PTX-OG+5 mg/kg CIP2b. After 24 hours, mice were euthanized, and tumors were collected and submerged in Hank's Balanced Salt Solution (HBSS) with calcium and magnesium. Tumors were then taken out of HBSS (one at a time to maintain cell viability), placed on a Petri dish, and mechanically minced with a sterile scalpel and forceps into small pieces. Tumor pieces were transferred to 50 mL tubes containing 10 mL of the pre-warmed (37° C.) sterile cell dissociation buffer (HBSS containing 50,000 U of collagenase IV and 100,000 U of DNase I). Samples were incubated at 37° C. under continuous rotation at 300 rpm for 60 minutes in the incubator-shaker. Then, cell suspensions were filtered through a 70 μm cell strainer then a 40 μm cell strainer to remove any aggregates. Subsequently, the filtrate (i.e., single-cell suspension) was centrifuged at 230 xg for 10 minutes. Cell pellets were then collected, resuspended in Ammonium-Chloride-Potassium (ACK) lysing buffer (deionized water containing 1% w/v of NH₄Cl, 0.125% w/v of KHCO₃, 0.25% v/v of 5% EDTA; pH 7.4) to lyse red blood cells, and incubated at room temperature for approximately 3 minutes. Cells were then collected and washed twice with 1× DPBS to remove free drug (i.e., extracellular drug). Finally, cells were resuspended in a complete medium containing 25 μg/mL PI to evaluate the cell viability after processing the tumors into a single cell suspension. Finally, cell suspensions were acquired using a FACSCalibur flow cytometer, and data were analyzed using FlowJo software.

Tumor volume (mm 3)=length (mm)×width (mm)×height (mm)×π/6 Equation 2:

Antitumor efficacy study following subcutaneous injection (SC) at the tumor site (peritumoral). Female athymic Nu/Nu mice were subcutaneously challenged with Hec50co cells (1×10⁶ cells/mouse). When mice had palpable tumors, they were randomly distributed into 3 groups: PBS group (n=3), PTX group (n=4), and PTX+CIP2b group (n=4). Then, treatments were administered subcutaneously near the tumor site (i.e., peritumorally) in five doses on days 26, 30, 33, 37, and 40 post-tumor challenge. Each dose consisted of 200 μL of either PBS, 5 mg/kg PTX (Paclitaxel concentrate diluted in DPBS), or 5 mg/kg PTX+5 mg/kg CIP2b (CIP2b in DPBS:Tween-80:ethanol 85:10:5; further dilution was made using DPBS). Body weights and tumor volumes (using Equation 2) were monitored twice a week for up to 2 weeks after the last dose (i.e., up to day 53 post-tumor challenge). On the last day of the study (day 53 post-tumor challenge), mice were euthanized, and tumors were collected and their weights were measured.

In vivo tumor efficacy study following intravenous injection (IV). Female athymic Nu/Nu mice were subcutaneously challenged with Hec50co cells (1×10⁶ cells/mouse). When mice had palpable tumors (day 18 post-tumor challenge), they were randomly distributed into 4 groups: PBS group (n=6), PTX+Blank NPs group (n=7), PTX+CIP2b solution group (n=7), and PTX+CIP2b NPs (n=7). Then, treatments were administered by IV injection in the tail vein in three doses on days 19, 23, and 26 post-tumor challenge. Each dose consisted of 200 μL of either PBS, 5 mg/kg PTX (Paclitaxel concentrate diluted in DPBS)+blank NPs (equivalent to the PLGA present in the CIP2b NPs used in one of the other treatment groups), 5 mg/kg PTX+5 mg/kg CIP2b (CIP2b in DPBS:Tween 80:ethanol 85:10:5; further dilution was made using DPBS), and 5 mg/kg PTX+CIP2b NPs (equivalent to 5 mg/kg CIP2b). Treatments that include CIP2b NPs or blank NPs were diluted with DPBS to the required volume. Body weights and tumor volumes (using Equation 2) were monitored twice a week for up to 4 weeks after the last dose (i.e., up to day 53 post-tumor challenge). On the last day of the study (day 53 post-tumor challenge), mice were euthanized, and tumors were collected and their weights were evaluated. Vital organs were also collected, and they, along with tumors, were fixed for histological and immunohistochemistry evaluation as described in Section 2.9.5. In addition, blood samples were also collected from euthanized mice to harvest serum, and sera samples were used to assess the safety of the treatments as described below.

Immunohistochemistry. To further assess the antitumor activity of the drug combination, immunohistochemistry (IHC) analysis was performed on the tumor tissues. On the last of the in vivo efficacy study (day 53 post tumor inoculation), representative tumors were randomly collected from the mice and washed with 1× DPBS. Tumors were then stored overnight in a 70% v/v ethanol 200 proof to rehydrate specimens. Subsequently, the ethanol was replaced by a 10% neutral buffered formalin until processing. Then, tumor specimens were processed into paraffin tissue blocks and followed by sectioning and mounting onto microscope slides. This was followed by staining with various stains including: (i) H&E to look at tumor microanatomy, (ii) CD31 to evaluate the degree of tumor angiogenesis, (iii) caspase 3 to assess apoptosis, (iv) β-tubulin to examine microtubule integrity, and (v) Ki67 to measure tumor cell proliferation and growth. Stained samples were visualized using a CKX41 Inverted Microscope equipped with DP70 Digital Camera System (Olympus, Japan). The scale bar was then added using ImageJ.

Safety study. On the last of the in vivo efficacy study (day 53 post tumor inoculation), 3 mice were randomly selected to study the safety of the treatments. Briefly, mice were anesthetized, and blood was collected from the mice via cardiac puncture. Blood samples were incubated for 30 minutes at room temperature, and this was followed by centrifugation for 15 minutes at 16,000 xg and 4° C. Serum samples (supernatants) were harvested to perform comprehensive analysis by measuring different enzyme levels (e.g., alanine aminotransferase ALT, alkaline phosphatase ALP, aspartate aminotransferase AST, creatine kinase CK) and other parameters (e.g., bilirubin BIL, blood urea nitrogen BUN, albumin, bicarbonate TCO₂, creatinine). Finally, these serum samples were used for the measurement of different parameters. Additionally, vital organs (heart, lungs, kidneys, spleen, and liver) from a representative mouse from each study group were collected to evaluate the histology. These organs were washed with 1× DPBS, then stored at room temperature in a 10% neutral buffered formalin (Research Products International). Then, samples were processed onto microscope slides as described above (Section 2.9.5) and stained with hematoxylin and eosin (H&E) stains. H&E-stained slides were imaged using a CKX41 Inverted Microscope equipped with DP70 Digital Camera System. The scale bar was then added using ImageJ.

Pharmacokinetics study. Female Balb-c mice were injected intravenously through the tail vein with either a solution of CIP2b in DPBS:Tween-80:ethanol 85:10:5 (1.25 mg/kg) or CIP2b NPs (1.25 mg/kg) in DPBS. After pre-determined time points (5, 15, 30, 60, 300, and 1440 minutes), mice were euthanized by CO₂ followed by cervical dislocation, and their blood was collected by cardiac puncture in heparinized tubes. Blood samples were centrifuged at 16,000 xg for 15 minutes at 4° C., and plasma was collected (supernatant). Then, 2 mL of ethyl acetate and 15 μL of the internal standard (IS) solution (50 μg/mL of 7-OH flavone in methanol) were added to 200 μL of plasma, and the mixture was vortexed for 5 minutes, centrifuged (16,000 xg, 5 minutes), and separated. The process was repeated, and a total of 4 mL of ethyl acetate per sample was completely evaporated under a steady nitrogen stream using TurboVap LV (Caliper LifeSciences) at room temperature for 90 minutes. Next, 150 μL of methanol and water mixture (1:1) were added to the residue, and the resultant dispersion was centrifuged (16,000 xg, 30 min). Finally, the clear supernatant was injected into the HPLC for quantification as described below (Section 2.9.9). A standard curve was constructed using blank mice plasma spiked with 15 μL of CIP2b solutions with different concentrations and 15 μL of the IS solution. The standard curve was linear over a range of 0-25 μg/mL (r²=0.9995).

In vivo tumor accumulation study of CIP2b following IV injection of CIP2b solution or CIP2b NPs. Athymic Nu/Nu mice were challenged with 1×10⁶ of Hec50co tumor cells per mouse on day zero of the experiment. Once the tumors became palpable, mice were injected IV through the tail vein with either a solution of CIP2b in DPBS:Tween 80:ethanol 85:10:5 (5 mg/kg) or CIP2b NPs (5 mg/kg) in DPBS. After pre-determined time points (2, 18, 24, and 36 hours), mice were euthanized by CO₂ followed by cervical dislocation, and the tumors were collected and stored at −80° C. To extract the drug, thawed tumors (on ice) were homogenized for 120 sec at speed #5 using a bead tissue homogenizer (Fisherbrand™ Bead Mill 4 Homogenizer) in screw-capped 2 mL tubes containing about 25 zirconia/silica disruption beads and 0.25 mL of DPBS each. Subsequently, 2 mL of ethyl acetate and 15 μL of the IS solution were added to the tissue homogenate, and CIP2b was extracted twice similar to the procedure described below (Section 2.9.9). Next, the total volume (4 mL) of ethyl acetate was evaporated to dryness under vacuum, and the residue was reconstituted as described in the PK study. Finally, the clear supernatant was injected into the HPLC for quantification. A standard curve was constructed using relevant blank mice tumor homogenate spiked with 15 μL of CIP2b solutions with different concentrations and 15 μL of the IS solution. The standard curve was linear over a range of 0-5 μg/mL (r²=0.9997).

Analytical analysis of CIP2b in biological samples using HPLC. A gradient elution method was adopted, and the mobile phase consisted of solution A (water+0.1% v/v trifluoroacetic acid (TFA)) and solution B (methanol:acetonitrile 95:5+0.1% v/v TFA). The gradient elution schedule started with solution A: solution B 90:10, then change gradually to 30:70 during the first 20 min, then remained at 30:70 for 2 more min, and finally change gradually to 90:10 during the last 3 min of the HPLC run. The same HPLC system, column, flow rate, injection volume, temperature, and wavelength were used as described above (Section 2.6.4).

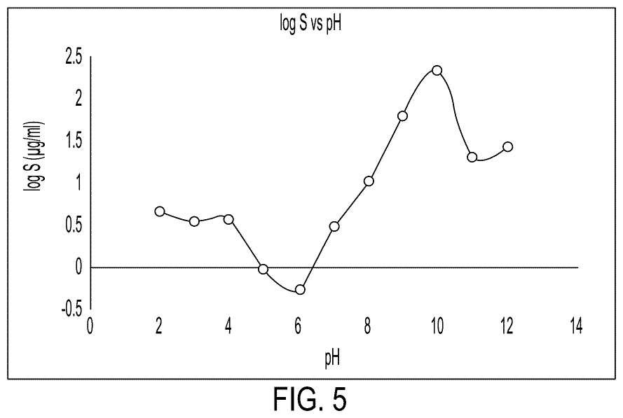

pKa determination. pKa was determined using the following solubility equation: log S=log Sₒ+log (10^{-pK}^{ₐ}^{+pH}+1)=(log Sₒ−pKₐ)+pH, where intercept=(log Sₒ−pKₐ) and slope=1 [1]. From the solubility (log S) vs pH profile graph based on the above equation, the intrinsic solubility was determined, log Sₒ, is −0.26 or 0.5μg/mL (FIG. 5). Two pKa values were found to be 5.4 and 6.0. There was another inflection point near pH 12, which could be another potential pKa.

Statistical analysis. In this study, data were compared either by two-tailed Student's T-test or by one-way analysis of variance (ANOVA) followed by Tukey post-hoc test to compare groups as deemed appropriate. Statistical analysis was performed using Prism 8 (GraphPad Prism, La Jolla, CA). Values with p<0.05 were considered statistically significant.

### Results and Discussion

### PTX and CIP2b Combination Exerts Synergistic Anti-Proliferative Activity Against Hec50co Cells and Enhances PTX Intracellular Accumulation

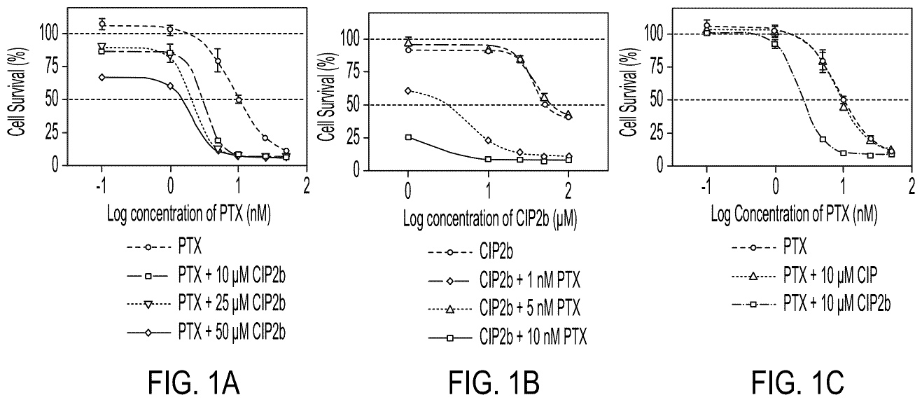

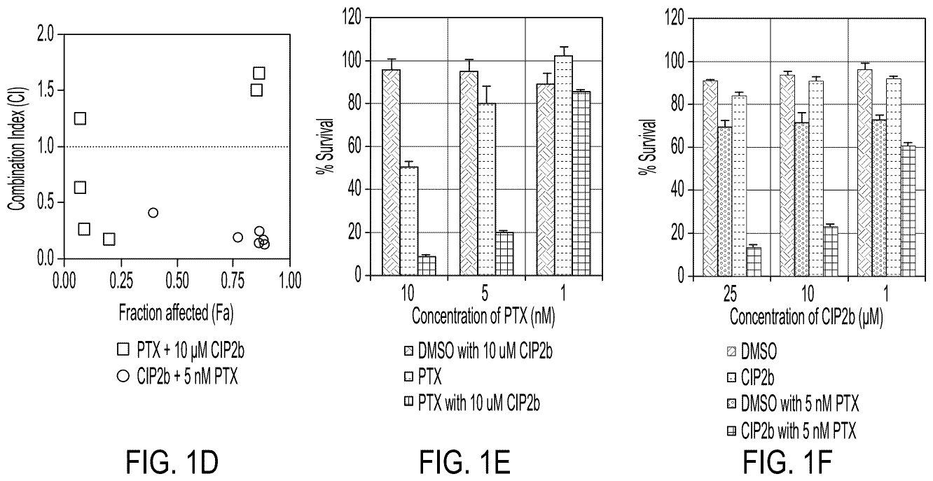

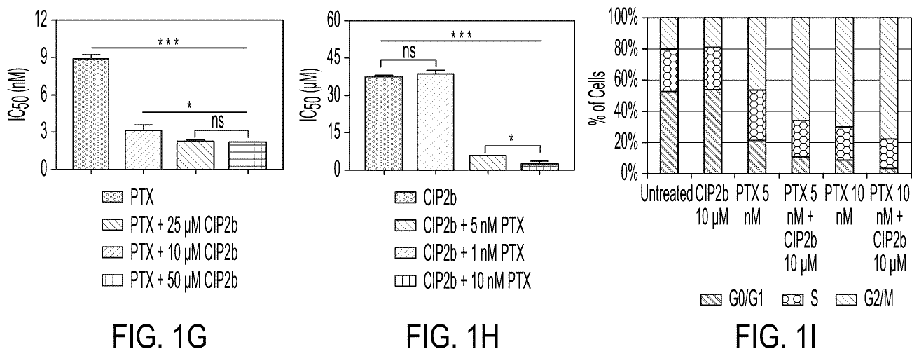

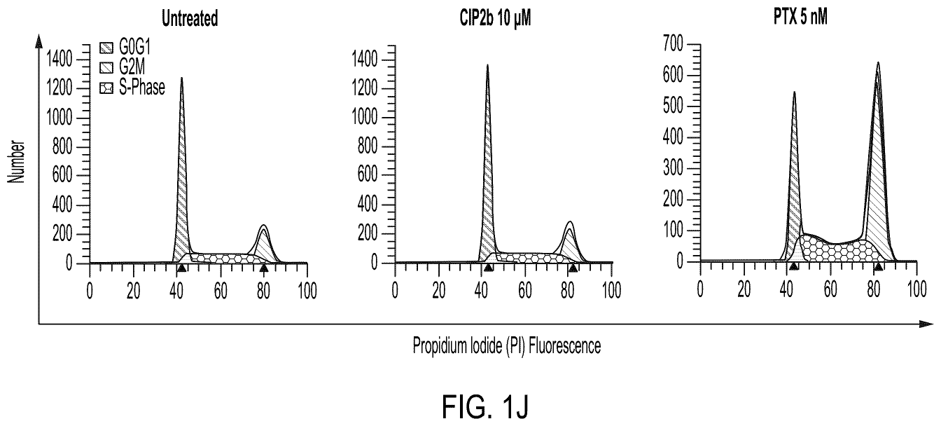

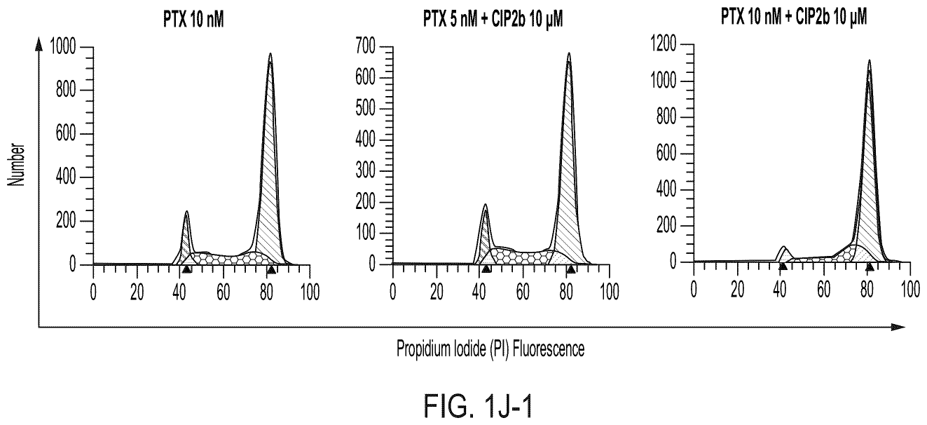

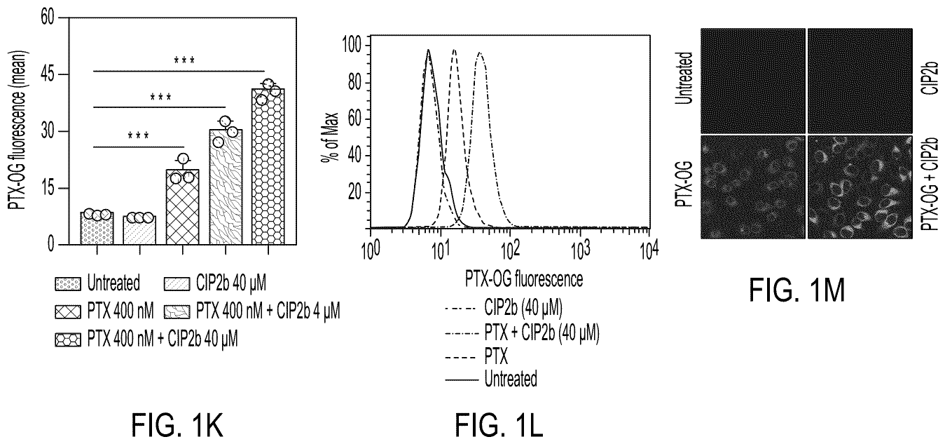

Cytotoxicity assay. Previous research showed that the CIP2b cytotoxicity against A549 lung cancer cells is dependent on p53 expression (Mohammed H H H, et. al., Bioorganic & medicinal chemistry. 2016; 24(19):4636-46, doi: 10.1016/j.bmc.2016.07.070). Our current research aimed to maximize the efficacy of chemotherapy against LOF endometrial cancer through the co-administration of novel adjuvant therapeutics with multiple mechanisms. As expected, CIP2b had a limited cytotoxic activity against the LOF p53 Hec50co cells as a monotherapy. However, the combination of CIP2b and PTX surprisingly and synergistically abolished cell survival, suggesting the presence of other mechanisms behind this improved activity. When CIP2b (10, 25, and 50 μM) was added to PTX, the Hec50co survival was reduced significantly in a dose-dependent manner (FIG. 1A). A similar trend was also found, and even to a larger extent, when PTX (1, 5, and 10 nM) was added to CIP2b (FIG. 1B). This was not the case when 10 μM CIP (the parent compound of CIP2b) was added to PTX, as the sigmoidal survival curves of PTX and PTX+10 μM CIP are superimposed (FIG. 1C). Calculations of the CI using Compusyn software revealed that most of the concentrations tested were synergistic (CI<1, FIG. 1D). Also, data showed an abrupt drop in cell survival when combinations were used (FIG. 1E and FIG. 1F). For example, when 5 nM PTX and 10 μM CIP2b were used as single compounds, the cell survival values were about 80% and 95%, respectively. However, there was a significant drop (p<0.001) in cell survival when a combination of these two compounds was used, reaching a value below 20% (FIG. 1E). Similarly, when 5 nM of PTX was added to 25 μM CIP2b, the cell survival was significantly (p<0.001) decreased from about 84% to about 14% (FIG. 1F). In addition, the IC₅₀ value of PTX was significantly decreased (p<0.01) from 8.77 nM to 3.1 nM and 2.15 nM when 10 and 25 μM CIP2b were added, respectively (FIG. 1G). Similarly, the IC₅₀ of CIP2b was significantly (p<0.01) decreased from 37 μM to 5.2 and 2.07 μM when 5 nM and 10 nM of PTX were added, respectively (FIG. 1H). These results demonstrated that both agents substantially amplify the effect of each other.

Cell cycle assay. Paclitaxel is known to promote microtubule polymerization and stabilize the microtubule assembly (Jordan M A, Wilson L. Nature reviews Cancer. 2004; 4(4):253-65, doi: 10.1038/nrc1317). It freezes cancer cells at the G2 and M phases of the cell cycle; hence, kills the dividing cells [5, 6]. Hec50co cell cycle analysis revealed that upon increasing the concentration of PTX from 5 nM to 10 nM, the cell population at the G2/M phase increased from approximately 46 to 70% (FIG. 1I and FIG. 1J). Also, the addition of 10 μM CIP2b to 5 nM PTX increased the cell population at the G2/M phase from 46% to 66.1%. Similar results were observed when 10 μM CIP2b was added to 10 nM PTX where the cell population at the G2/M phase reached 77.4% compared to 70% when 10 nM PTX was tested. CIP2b itself (10 μM) had an unnoticeable effect on the Hec50co cell cycle.

PTX intracellular accumulation. PTX-OG was used to assess whether CIP2b enhances the intracellular accumulation of PTX by quantitative and qualitative methods. PTX was readily taken up by Hec50co cells, as clearly shown in FIG. 1K. CIP2b itself had no fluorescence of its own where CIP2b treated cells did not exhibit any shift in comparison to untreated cells. The addition of 4 μM CIP2b to cells significantly enhanced the cellular accumulation of PTX compared to untreated cells (p<0.0001) and to PTX alone (p<0.001). This effect was dose-dependent enhancement where PTX+40 μM CIP2b showed significantly higher intracellular accumulation of PTX compared to PTX+4μM CIP2b (p<0.001). As shown in FIG. 1L, the peak was completely shifted when CIP2b was added to PTX. These findings were also supported by the confocal microscopy images (FIG. 1M). As expected, treating Hec50co cells with CIP2b did not show any fluorescence while PTX was readily taken up by Hec50co cells and labeled the tubulin filaments in the live Hec50co cells when compared to untreated cells. Interestingly, CIP2b enhanced PTX accumulation in Hec50co cells as evidence by the increase in the fluorescence intensity.

### CIP2b Improves PTX Intracellular Accumulation, Enhances the Apoptotic Activity and Tubulin Stabilization of PTX, and Inhibits Topoisomerase I & II

Caspase expression. Caspases are cysteine proteases that regulate apoptosis. Executioner caspases like caspase 3 and 6 are usually activated following the activation of upstream initiator caspases (like caspase 8 and 9) (Degterev A, Boyce M, Yuan J. Oncogene. 2003; 22(53):8543-67, doi: 10.1038/sj.onc.1207107). Caspase 8 is activated following an extrinsic pathway via stimulation of death receptors like TNF-α related apoptosis stimulating ligand (TRAIL), while caspase 9 is activated by an intrinsic pathway through the mitochondria (Fulda S, Debatin K M. Oncogene. 2006; 25(34):4798-811, doi: 10.1038/sj.onc.1209608). The mitochondrial pathway involves the release of cytochrome c into the cytosol and the subsequent formation of apoptosome complex which trigger caspase-3 activation, while other released factors like Smac/DIABLO counteract the apoptosis inhibitory activity of the anti-apoptotic proteins (IAPB) (inhibitors of apoptosis, including XIAP and survivin) (Fulda S, Debatin K M. Oncogene. 2006; 25(34):4798-811, doi: 10.1038/sj.onc.1209608; MacKenzie S H, Clark A C. Current cancer drug targets. 2008; 8(2):98-109, doi: 10.2174/156800908783769391; and LaCasse E C, et. al., Oncogene. 2008; 27(48):6252-75, doi: 10.1038/onc.2008.302). Chemotherapeutics may induce apoptosis through either pathway.

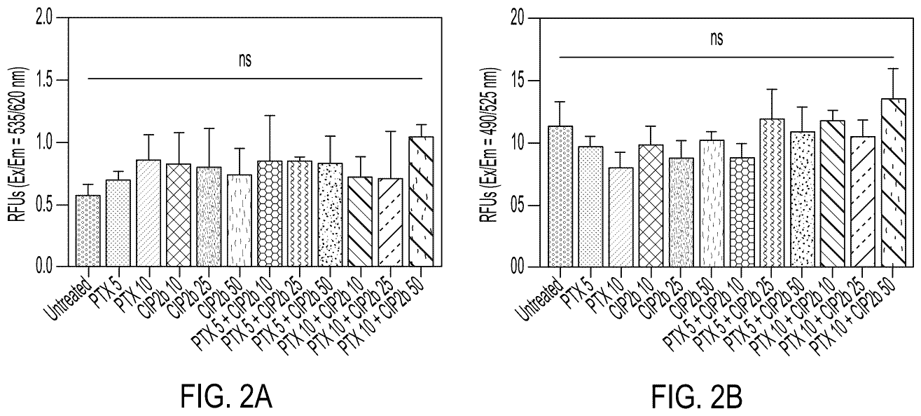

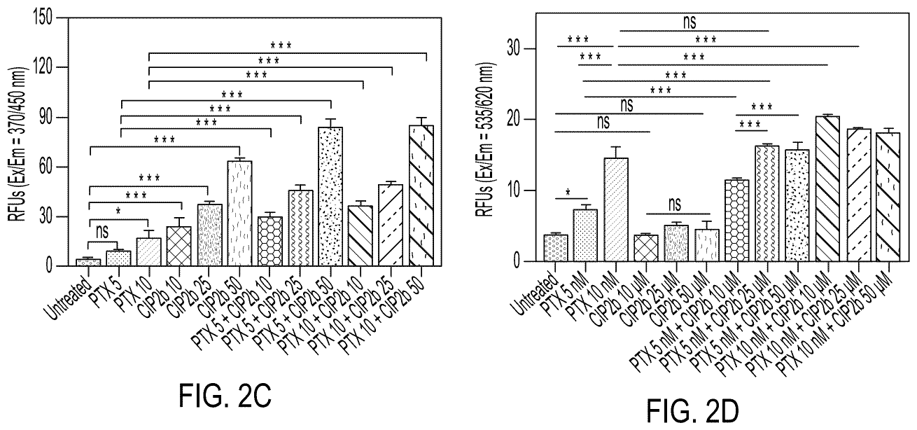

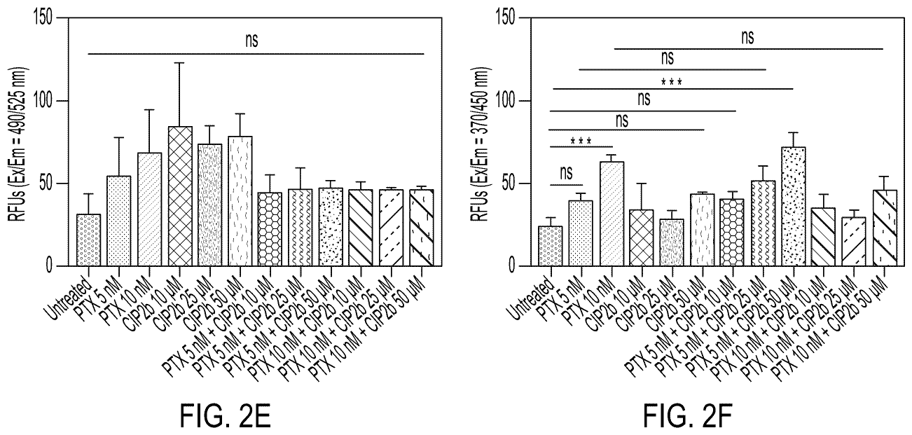

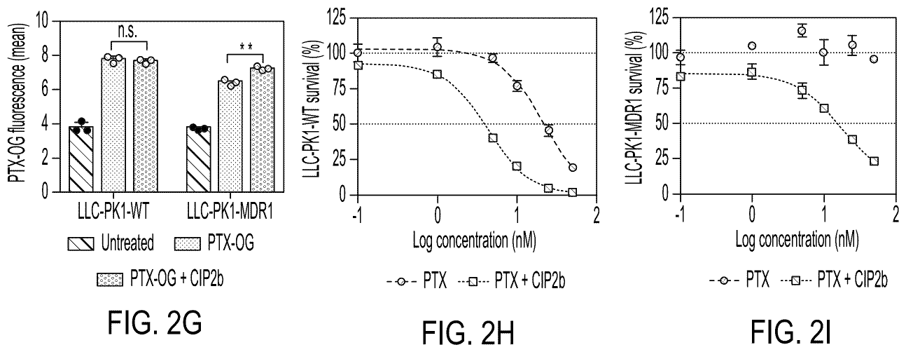

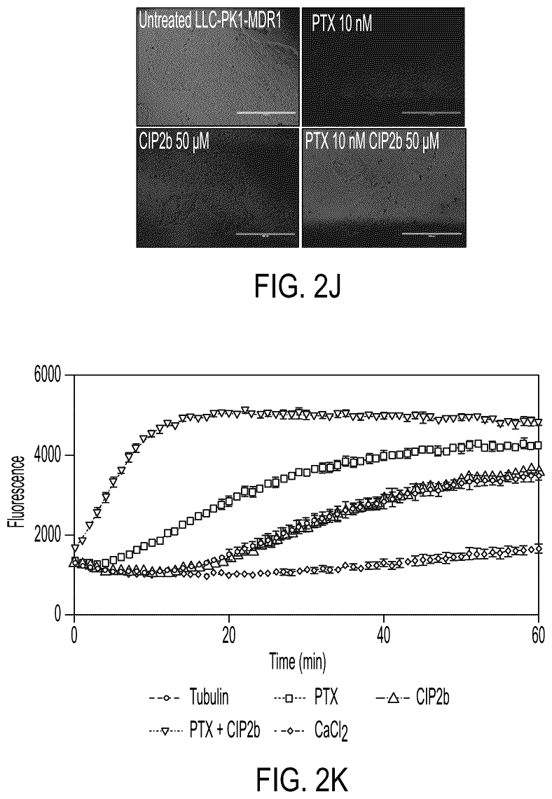

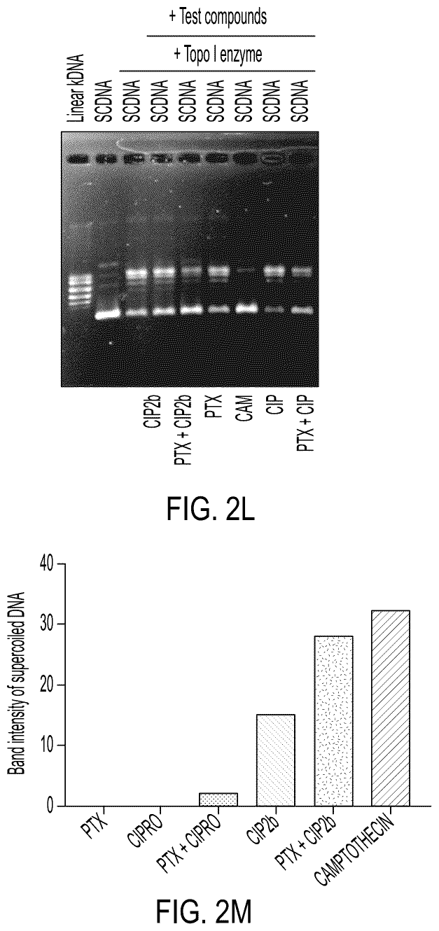

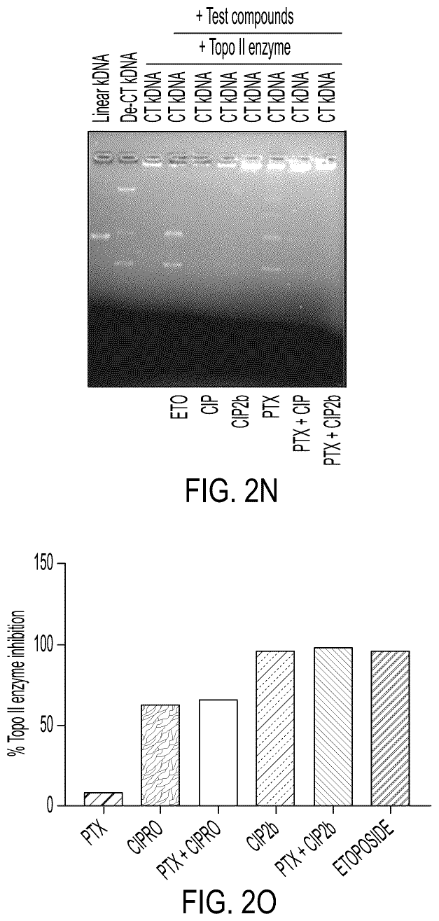

To identify which pathway leads to apoptosis following the treatment of Hec50co cells with CIP2b, PTX, or their combinations, different proteolytic caspases were studied at different time points (24 and 72 h, FIG. 2A-F). After 24 hours of incubation with the treatments, it was found that effector caspase levels (caspase 3) did not show any significant changes (FIG. 2A), and similarly, there was no significant difference in the level of caspase 8 (FIG. 2B), indicating no involvement of the intrinsic pathway or death receptors. However, the level of caspase 9 expression (FIG. 2C) was significantly higher in the cells treated with 10 nM of PTX compared to the untreated group (p<0.05) whereas the expression of caspase 9 in Hec50co cells treated with a low dose of PTX (i.e., 5 nM) was not statistically different from untreated cells. Additionally, the expression of caspase 9 of CIP2b treated cells was significantly higher than the untreated group at all tested concentrations (i.e., 10, 25, 50 μM) and in a concentration-dependent manner. Interestingly, co-delivery of CIP2b with PTX significantly enhanced the apoptotic effect compared to untreated as well as PTX treated cells. For example, cells treated with 5 nM PTX and any CIP2b concentration (i.e., 10, 25, 50 μM) resulted in a significant increase in the caspase 9 expression compared to 5 nM treated cells and untreated cells. Even though 5 nM PTX did not induce significant activation of caspase 9 in cells, 5 nM PTX+50 μM CIP2b induced caspase 9 activation significantly higher than 50 μM CIP2b. After 72 h of adding the treatments (FIG. 2D-F), there was a significant increase in the expression of caspase 3 (FIG. 2D). Particularly, cells treated with PTX displayed high levels of caspase 3 compared to untreated cells, and these levels were further boosted when CIP2b was co-delivered with PTX. CIP2b did not induce any activation of caspase 3 after 72 hours, even though it showed some activation of caspase 9 after 24 hours (FIG. 2C), while none of the treatments induced any substantial changes of caspase 8 levels (FIG. 2E). Finally, caspase 9 levels in PTX+CIP2b treated cells either did not change or have decreased (FIG. 2F) compared to the 24 hour time point (FIG. 2C). Since caspase 9 is an initiator caspase, it is expected to increase in level at earlier time points, then decrease later on. On the contrary, caspase 3, being an executioner end-stage caspase, should rise in level with time. This is in agreement with what was reported by Bozec et al., who studied the time dependence of apoptosis following finasteride administration to patients for the treatment of benign prostatic hyperplasia (BPH) (Bozec A, et. al., The Journal of clinical endocrinology and metabolism. 2005; 90(1):17-25, doi: 10.1210/jcem.90.8.9993). Bozec et al. found that the levels of cleaved caspase 3 and 6 (executioner caspases) did not show a significant rise compared to the untreated specimen after earlier time points (3 days). However, their levels rose sharply after 7 days. The opposite pattern was shown by cleaved caspase 9 levels, as its level showed a significant rise after 3 days, then it markedly declined after 7 days. Meanwhile, even though CIP2b resulted in an early rise of caspase 9 levels in Hec50co cells, this did not result in a corresponding rise in caspase 3 levels, which may be due to the activity of IAPB, especially in LOF p53 cells like Hec50co. It is clear that the addition of PTX reversed that resistance effect and switched on the cytotoxic activity of CIP2b, as clearly shown in the ICso values (FIG. 1H).

PTX accumulation in LLC-PKI-WT and LLC-PKI-MDR1 cells. P-gp's are membrane-bound proteins that belong to an evolutionarily conserved family of ATP-binding cassette (ABC) protein transporters (Sharom F J. Pharmacogenomics. 2008; 9(1):105-27, doi: 10.2217/14622416.9.1.105; De Vera A A, et. al., Cancer letters. 2019; 442:91-103, doi: 10.1016/j.canlet.2018.10.020; and Sarkadi B, et. al., Physiological reviews. 2006; 86(4):1179-236, doi: 10.1152/physrev.00037.2005). Overexpression of P-gp in cancer patients is linked with poor prognosis and short overall survival (Zhang Y, et. al., Molecular cancer therapeutics. 2016; 15(10):2282-93, doi: 10.1158/1535-7163.MCT-15-0986), and its targeting remains one of the most efficient pathways to reduce chemo-resistance. PTX, among other agents that bind to tubulin, is a substrate of P-gp, and its energy-dependent P-gp-mediated efflux from cancer cells, even after reaching tumors, significantly shortens the time it spends inside the cells. This prevents its accumulation inside the cells, which in turn markedly reduces its efficacy. The use of P-gp inhibitors for the reversal of PTX P-gp-mediated resistance (e.g. the 3^{rd} generation inhibitor tariquidar) in pre-clinical (including those involving nano-carriers (Zhang Y, et. al., Molecular cancer therapeutics. 2016; 15(10):2282-93, doi: 10.1158/1535-7163.MCT-15-0986; and Patil Y, et. al., Journal of controlled release. 2009; 136(1):21-9, doi: 10.1016/j.jconre1.2009.01.021) and clinical (Kelly R J, et. al., The oncologist. 2012; 17(4):512, doi: 10.1634/theoncologist.2012-0080) settings is widely described. To find out whether this enhancement of uptake is related to MDR1 (P-gp), we used the wild type of LLC-PK1 (LLC-PK1-WT) cells and their MDR1-overexpressing variant LLC-PK1-MDR1.

Flow cytometry-based cellular uptake analysis showed that CIP2b significantly enhanced the intracellular accumulation of PTX in MDR1-overexpressing cells only (p<0.05) while there was no difference between the two treatments in WT cells (FIG. 2G). This clearly shows that the improved cellular uptake is strongly related to P-gp expression. Two possible explanations can stand behind this observation. The first one is that CIP2b blocks the P-gp efflux and, in turn, results in more accumulation of PTX in the cells. The second one is that CIP2b is also a P-gp substrate and competitively competes with PTX. We sought to check whether CIP2b fulfills the rules of P-gp substrates (Didziapetris R, et. al., Journal of drug targeting. 2003; 11(7):391-406, doi: 10.1080/10611860310001648248; and Singh S, et. al., Journal of medicinal chemistry. 2014; 57(10):4058-72, doi: 10.1021/jm401966m). Generally, a drug molecule is considered a P-gp substrate if it has: (i) a total number of nitrogen and oxygen atoms≥8, (ii) molecular weight>400 g/mol, and (iii) acidic pKa>4 (Didziapetris R, et. al., Journal of drug targeting. 2003; 11(7):391-406, doi: 10.1080/10611860310001648248; and Singh S, et. al., Journal of medicinal chemistry. 2014; 57(10):4058-72, doi: 10.1021/jm401966m). According to these rules, CIP2b is potentially a P-gp substrate since it has 8 atoms of combined nitrogen and oxygen, a molecular weight of 497 g/mol, and two acidic pKa values of 5.4 and 6 (FIG. 5). This was further supported by the molecular interaction of CIP2b with P-gp.

The cytotoxicity of CIP2b and PTX combination against LLC-PK1-WT cells was much higher compared to PTX alone as shown in FIG. 2H (IC₅₀ of 4.3 nM compared to 21.3 nM, respectively). Although there was no difference in the intracellular accumulation of PTX between the two treatment groups (FIG. 2G), the superiority of cytotoxicity of PTX+CIP2b in LLC-PK1-WT cells compared to PTX alone (FIG. 2H) suggests that there is another mechanism(s) of action by which CIP2b enhances the antitumor activity of PTX. While PTX was not effective at all up to the maximum concentration used (50 nM) against LLC-PK1-MDR1 (no IC₅₀ reached), the combination was still very effective against the MDR1-overexpressing cells as shown in FIG. 2I (IC₅₀ of 16.8 nM). These results were further supported by images from bright field microscopy and utilizing a 10× objective lens (FIG. 2J). In the images, rounded cells (morphological change) represent cells undergoing apoptosis (i.e., under stress), and this was more obvious in the PTX+CIP2b treated group.

Tubulin polymerization assay. The results from the tubulin polymerization assay showed that tubulin required 15 min to start polymerization (i.e., the onset of reaction, FIG. 2K). When PTX was added, the rate of polymerization was promoted compared to tubulin polymerization in the absence of any treatment. Also, the onset of tubulin polymerization became shorter (approximately 5 minutes) when PTX was added. When CIP2b alone was tested, there was no effect. Surprisingly, when CIP2b was added to PTX, there was a significant enhancement in the rate of tubulin polymerization. Additionally, it was observed that tubulin polymerization took place immediately (the onset of the reaction was almost zero, FIG. 2K). These results clearly demonstrate that CIP2b promotes the microtubule stabilization activity of PTX.

PTX enhances the rate of tubulin polymerization and stabilizes the formed microtubules, though both functions take place at different dynamics. In fact, microtubule stabilization needs very few PTX molecules, in contrast to tubulin polymerization, which, at a stoichiometric ratio of 1:1 of PTX:tubulin, needs thousands of PTX molecules to bind a sufficient number of tubulin molecules and form the microtubule (assuming that it is composed of several thousands of tubulin molecules) (Jordan M A, Wilson L. Nature reviews Cancer. 2004; 4(4):253-65, doi: 10.1038/nrc1317). PTX is thought to inflict conformational changes to tubulin molecules which propagate their polymerization reaction (Jordan M A, Wilson L. Nature reviews Cancer. 2004; 4(4):253-65, doi: 10.1038/nrc1317; and Hangas A, et. al., Nucleic acids research. 2018; 46(18):9625-36, doi: 10.1093/nar/gky793). The extensively faster tubulin polymerization rate when CIP2b was added to PTX compared to PTX alone suggests that CIP2b helps to accelerate the kinetics of the binding of PTX to its specific sites in tubulin molecules.

Topoisomerase inhibition I assay. Results obtained from the topoisomerase I assay demonstrated that SC DNA displayed one bright band as shown in lane 2 (FIG. 2L). When human Topo I was added as shown in lane 3, SC DNA was converted to relaxed DNA and, thus, displayed multiple bands (similar to relaxed DNA shown in lane 1) with a discreet faint band at the end of the run. CAM is a potent inhibitor of Topo I, and thus the DNA in its band remains supercoiled and appears as one bright band at the end of the run (lane 7). It is clear that neither PTX nor CIP did inhibit Topo I as shown in lanes 6 and 8, respectively, and therefore the enzyme was able to convert the SC DNA to relaxed DNA, and thus, there was a faint band at the end of the run similar to lane 3. In contrast, in lane 1, multiple bands with a bright band (at the end of the run) were observed; indicating that CIP2b provided some Topo I inhibitory activity. Interestingly, when PTX was added to CIP2b as shown in lane 5, the magnitude of enzyme inhibition was increased as observed by the increase in the intensity of the band at the end of the run which was comparable to that of CAM (FIG. 2M). This is another evidence that both compounds benefit from each other, as PTX promotes the Topo I inhibitory activity of CIP2b while CIP2b promotes the PTX activity on tubulin polymerization and microtubule assembly.

Topoisomerase inhibition II assay. Topoisomerase II assay displayed that De-CT DNA had multiple bright bands (lane 2, FIG. 2N) whereas CT DNA did not exhibit any bands (lane 3). Upon adding the human Topo IIα enzyme to the catenated DNA, the enzyme converted the catenated rings to the decatenated form as shown in lane 4. As expected, ETO, the positive control, inhibited the Topo II enzyme as evidenced by the disappearance of bands (lane 5), and the percentage of enzyme inhibition was approximately 96% (FIG. 2O). CIP, the parent compound, exhibited some partial inhibition of Topo II (62%), which is consistent with recent findings [7]. Surprisingly, CIP2b achieved strong enzyme inhibition (96%) and was similar to that of ETO. As anticipated, PTX had a negligible effect on the enzyme inhibition (8%). Therefore, adding PTX to either CIP or CIP2b did not help in enhancing the enzyme inhibition compared to CIP and CIP2b alone where CIP+PTX and CIP2b+PTX result in 66% and 98% of Topo II inhibition, respectively (FIG. 2O).

CIP, as an antibiotic, acts by inhibiting prokaryotic Topo II (DNA gyrase and Topo IV) (Hooper D C, Jacoby G A. Cold Spring Harbor perspectives in medicine. 2016; 6(9), doi: 10.1101/cshperspect.a025320). Despite its high specificity against bacterial topoisomerases, recent work highlighted that they may also inhibit human Topo II when used in sufficient concentration (Hangas A, et. al., Nucleic acids research. 2018; 46(18):9625-36, doi: 10.1093/nar/gky793). Chemical modifications of the CIP backbone either at 1-N (Oppegard L M, et. al., Investigational new drugs. 2019; 37(2):378-83, doi: 10.1007/s10637-018-0666-x; and Delgado J L, et. al., European journal of medicinal chemistry. 2019; 172:109-30, doi: 10.1016/j.ejmech.2019.03.040) or 4-N (Mohammed H H H, et. al., Bioorganic & medicinal chemistry. 2016; 24(19):4636-46, doi: 10.1016/j.bmc.2016.07.070; Mohammed H H H, et. al., Bioorganic chemistry. 2021; 106:104422, doi: 10.1016/j.bioorg.2020.104422; and Abdel-Aziz M, et. al., European journal of medicinal chemistry. 2013; 69:427-38, doi: 10.1016/j.ejmech.2013.08.040) position impart strong inhibitory activity of human Topo I, II, or both and subsequently anticancer activity. A combination of topoisomerase inhibitors and PTX has been utilized to improve the anticancer activity and minimize chemo-resistance by the dual mechanisms. Some of these topoisomerase inhibitors potentiate other chemotherapeutics by several mechanisms. For example, Parmakhtiar et al. found that the hypoxia-inducible factor-1α, which helps ovarian carcinoma cells survive under hypoxic conditions, inhibits p53-mediated apoptosis, and is associated with an increased ABCB1 surface expression. When the cells are treated with topotecan, a potent Topo I inhibitor, it binds to Topo I on the HIF mRNA, blocking its translational, restoring p53 activity, and downregulating ABCB1 expression, which collectively reverses paclitaxel resistance in ovarian cancer cells (Parmakhtiar B, et. al., Molecular cancer research. 2019; 17(8):1675-86, doi: 10.1158/1541-7786.MCR-18-1109). Other combinations comprising topoisomerase inhibitors and PTX are in clinical trials (e.g. PTX, topotecan, and carboplatin for metastatic endometrial cancer (Papadimitriou C A, et. al., Gynecologic oncology. 2008; 111(1):27-34, doi: 10.1016/j.ygyno.2008.06.001) and irinotecan and PTX for non-small cell lung cancer (NSCLC) (Stathopoulos G P, et. al., British journal of cancer. 2005; 93(10):1106-11, doi: 10.1038/sj .bj c.6602827).

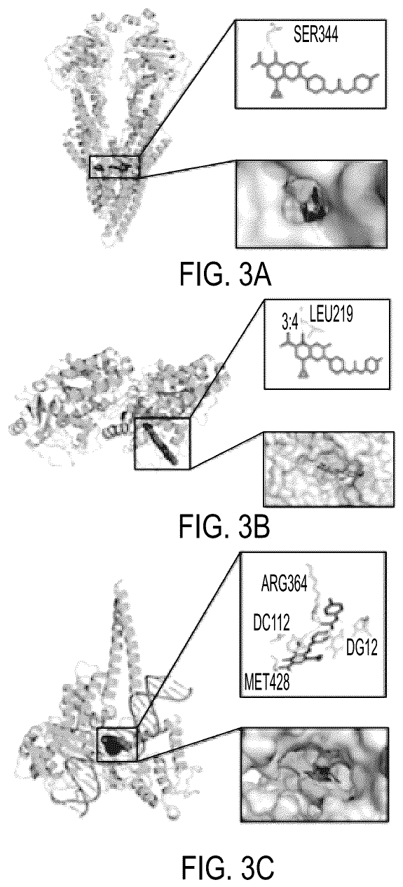

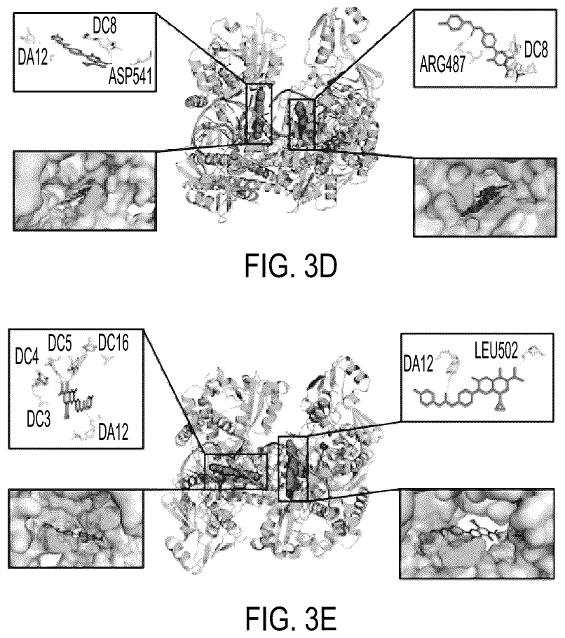

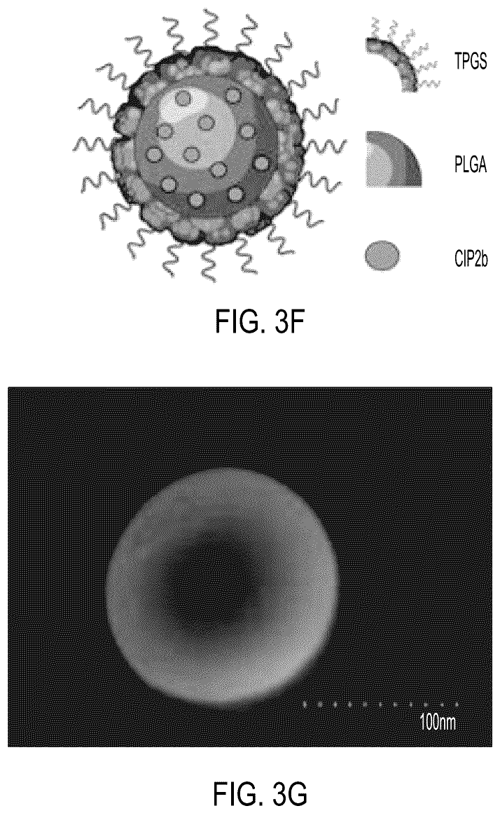

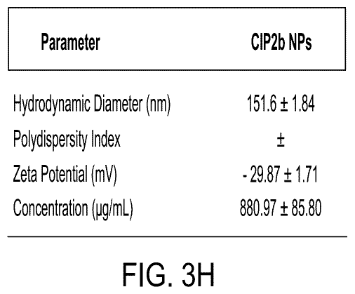

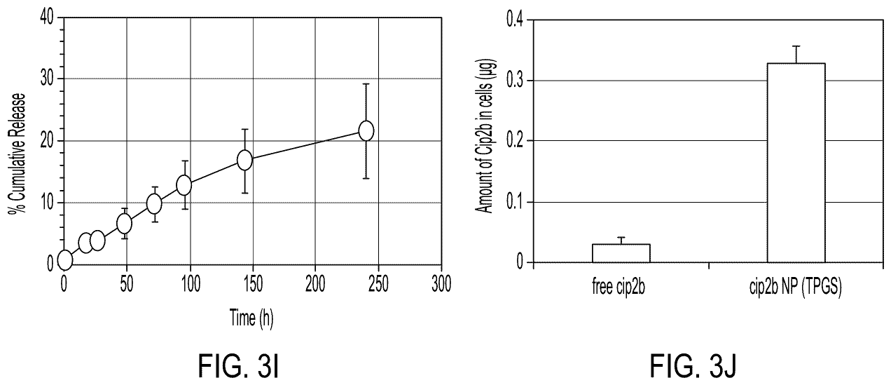

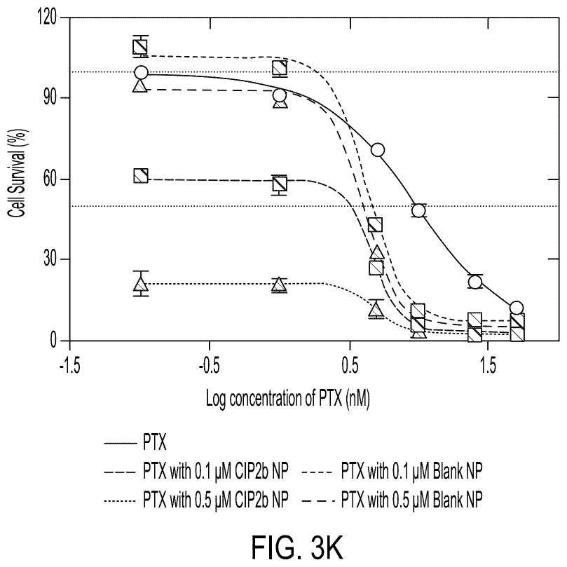

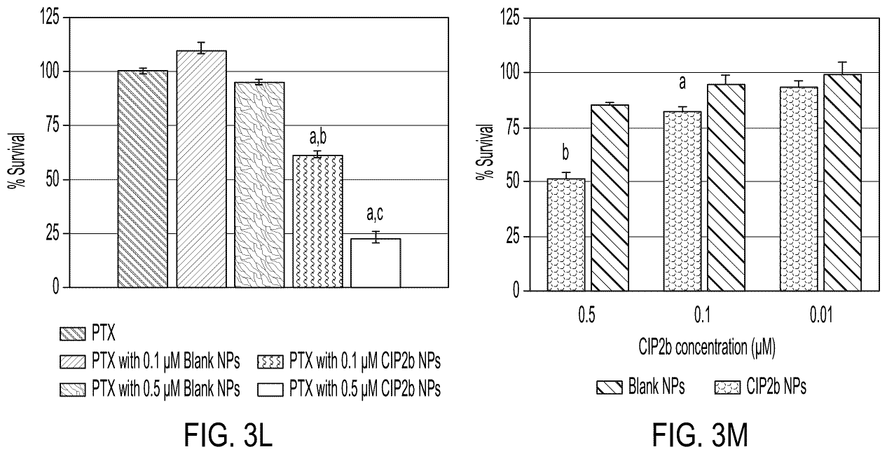

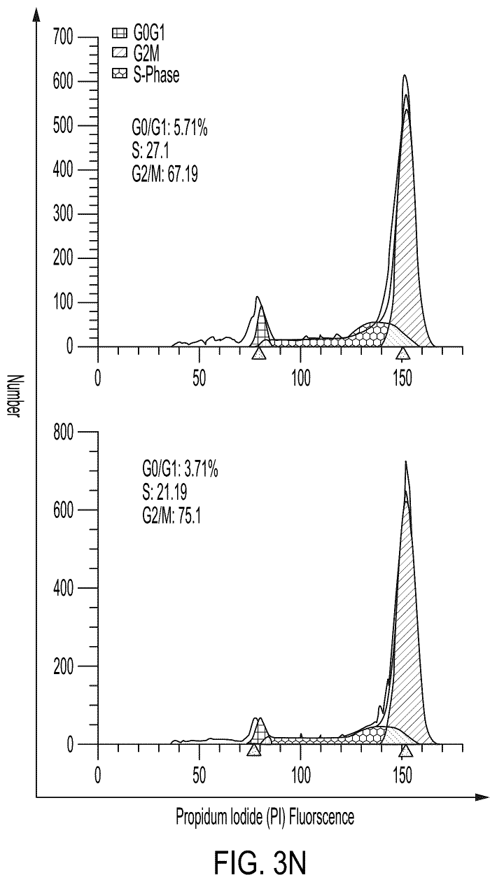

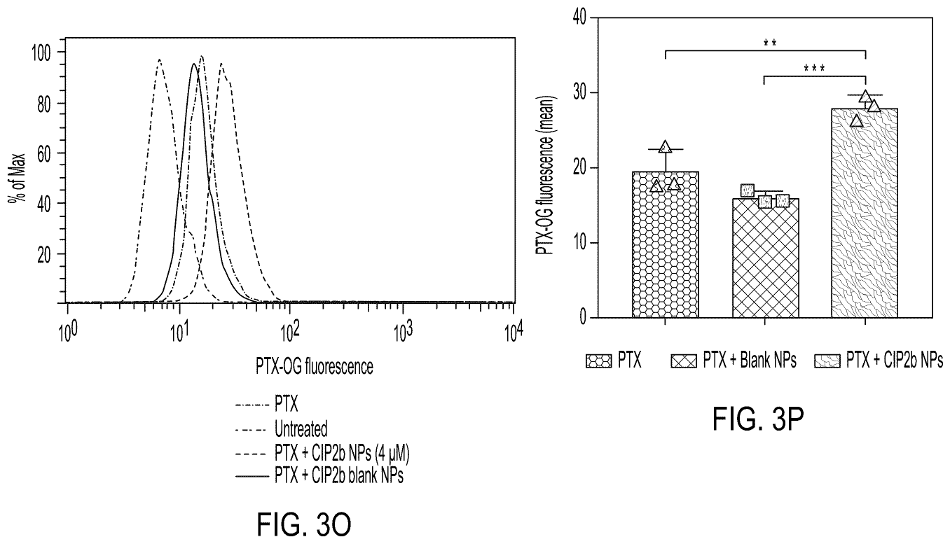

Molecular docking analysis. The activity of CIP2b was further confirmed by exploring the molecular interaction between CIP2b and protein molecules including human P-gp, tubulin, Topo I, and Topo II. C CIP2b showed a strong binding affinity towards all the protein targets with binding free energies ranging from −12.9 to -9.6 kcal/. For example, CIP2b exhibited a strong binding affinity to the paclitaxel binding site of the P-gp and interacted with several P-gp residues (e.g., SER344, FIG. 3A). In addition, CIP2b strongly interacted with the paclitaxel interaction site of αβ-tubulin stabilizing tubulin polymerization and interacted with different αβ-tubulin residues like LEU219 (FIG. 3B). Simulation of the molecular interaction of CIP2b with human Topo I displayed that CIP2b can interact with various Topo I residues such as DC112 and ARG364 (FIG. 3C). Molecular docking analysis also indicated that CIP2b had a strong binding affinity to human Topo IIα and Topo IIβ where CIP2b interacted with diverse Topo II residues (FIG. 3D and 3E). For instance, CIP2b interacted with DC8, DA12, and ARG487, which represent the binding sites for ETO on Topo IIα and Topo IIβ. Overall, CIP2b interacted with active site residues of all the targets.

### CIP2b NPs Exhibit Improved Cytotoxicity and Cellular Uptake in Endometrial Cancer Cells

CIP2b NPs preparation and characterization. CIP2b NPs were prepared using the nanoprecipitation method, and the proposed structure of the NPs is illustrated in a schematic diagram (FIG. 3F). Scanning electron photomicrograph of CIP2b NPs exhibited that NPs appeared to be spherical with smooth surfaces (FIG. 3G). Dynamic light scattering measurements displayed that CIP2b NPs had an average particle size of 151.6 nm, a net surface charge of about −29 mV, and drug loading of about 881 μg/mL (FIG. 3H). In vitro cumulative release of CIP2b from the NPs was slow, reaching about 22% by day 10 day of the release experiment (FIG. 3I). A zeta potential value of or around +or −30 mV helps maintain the NPs stability in colloidal suspension and prevents their aggregation (Sloat B R, et. al., International journal of pharmaceutics. 2011; 409(1-2):278-88, doi: 10.1016/j.ijpharm.2011.02.037). According to the Enhanced Permeation and Retention (EPR) phenomenon, tumor vasculature is formed of ill-formed leaky blood vessels that were developed in a haste in order to quickly fulfill the high nutritional demand of the rapidly proliferating tumor tissue (Fang J, et. al., Advanced drug delivery reviews. 2020; 157:142-60, doi: 10.1016/j.addr.2020.06.005; Fang J, Nakamura H, Maeda H. T Advanced drug delivery reviews. 2011; 63(3):136-51, doi: 10.1016/j.addr.2010.04.009; Maeda H. Journal of personalized medicine. 2021; 11(3), doi: 10.3390/jpm11030229; Maeda H. Journal of drug targeting. 2017; 25(9-10):781-5, doi: 10.1080/1061186X.2017.1365878; and Maeda H. Advanced drug delivery reviews. 2015; 91:3-6, doi: 10.1016/j.addr.2015.01.002). These tumors lack lymphatic drainage, and nanocarriers of size range below 200 nm are usually trapped within these tumors, while other soluble drugs are not (Naguib Y W, Cui Z. Advances in experimental medicine and biology. 2014; 811:207-33, doi: 10.1007/978-94-017-8739-0_11). Consequently, the size of the prepared CIP2b NPs is suitable to achieve the desired extravasation into the tumor tissue. In addition to the enhancement of tumor accumulation, formulating CIP2b into polymeric PLGA NPs will also help deliver the drug in a clinically feasible formulation ready for IV injection, since CIP2b is a newly developed compound, and no formulation has been reported to deliver it in vivo. A third advantage of using NPs to deliver CIP2b is to prolong its circulation time in order to evade the components of the mononuclear phagocyte system (Naguib Y W, Cui Z. Advances in experimental medicine and biology. 2014; 811:207-33, doi: 10.1007/978-94-017-8739-0_11). In this regard, TPGS was used as a surfactant in the NPs preparation as it inserts itself in the surface of the PLGA NPs via its lipophilic side (tocopheryl side) while its hydrophilic PEG chains are coating the NPs' outer surfaces rendering them hydrophilic, stealthy, and capable of evading opsonization and early uptake by macrophages (Kulkarni S A, Feng S S. Pharmaceutical research. 2013; 30(10):2512-22, doi: 10.1007/s11095-012-0958-3; Feng S S, et. al., Biomaterials. 2009; 30(19):3297-306, doi: 10.1016/j.biomaterials.2009.02.045; and Zhang Z, Tan S, Feng S S. Biomaterials. 2012; 33(19):4889-906, doi: 10.1016/j.biomaterials.2012.03.046). On the other hand, the slow release in physiological pH is a requirement to ensure that the loaded dose is not prematurely dumped in plasma before reaching the tumor.

Cellular uptake and cytotoxicity of CIP2b NPs. Results displayed that TPGS-decorated CIP2b NPs were more readily internalized by Hec50co cells in comparison to soluble CIP2b as evidenced by a significant increase of CIP2b intracellular accumulation (FIG. 3J). In addition, the cytotoxicity of Hec50co when CIP2b NPs were added to PTX was much higher than that obtained when soluble CIP2b was added to PTX (FIG. 3K). Results also showed that CIP2b NPs significantly augmented the cytotoxicity of PTX in comparison to the effect of blank NPs (FIG. 3L). For example, while the Hec50co survival was 100% when 0.1 nM of PTX was used alone and it did not change when blank NPs (equivalent to 0.1 or 0.5 μM CIP2b loaded NPs) was added (reaching about 109% and 95%, respectively), the survival of Hec50co cells treated with a combination of 0.1 nM PTX and 0.1 μM or 0.5 μM CIP2b NPs dropped significantly to 61.2% (p<0.01 compared to 0.1 nM PTX+0.1 μM blank NPs) and 21.3% (p<0.01 compared to 0.1 nM PTX+0.5 μM blank NPs), respectively. The concentrations used of blank NPs were equivalent to the same amount of CIP2b NPs loaded with the denoted CIP2b concentration. It is worth mentioning that the CIP2b NPs showed a dose-dependent improvement of cytotoxic effect against Hec50co cells, as the survival of cells declined from 92.7% to 81.5% and 50.5% when the concentration increased from 0.01 to 0.1 and 0.5 μM, respectively, whereas the corresponding concentrations of blank NPs did not show a similar decline (FIG. 3M). Furthermore, when 0.5 μM CIP2b NPs were added to 10 nM PTX, the cell population at the G2/M phase reached 75.1% instead of 67.2% when a combination of PTX+blank NPs (corresponding to 0.5 μM CIP2b loaded NPs) was used (FIG. 3N) or 69.5% when 10 nM PTX alone was tested (FIG. 1I). The CIP2b uptake into Hec50co cells was improved significantly (p<0.0001) by about 10-fold when CIP2b NPs were used instead of CIP2b solution (FIG. 2I), which highlights another advantage of using the NPs over the soluble drug. Additionally, the effect of soluble CIP2b on the intracellular accumulation of PTX in Hec50co cells was similarly observed with CIP2b NPs (FIG. 3O and FIG. 3P) where PTX+4μM CIP2b NPs group showed significantly higher accumulation of PTX compared to PTX (p<0.01) and PTX+blank NPs (p=0.001).

### CIP2b Co-Administered With PTX Significantly Improves the in Vivo Tumor Accumulation of PTX and Enhances the Antitumor Efficacy of PTX Against Human Endometrial Cancer Xenografts in Mice

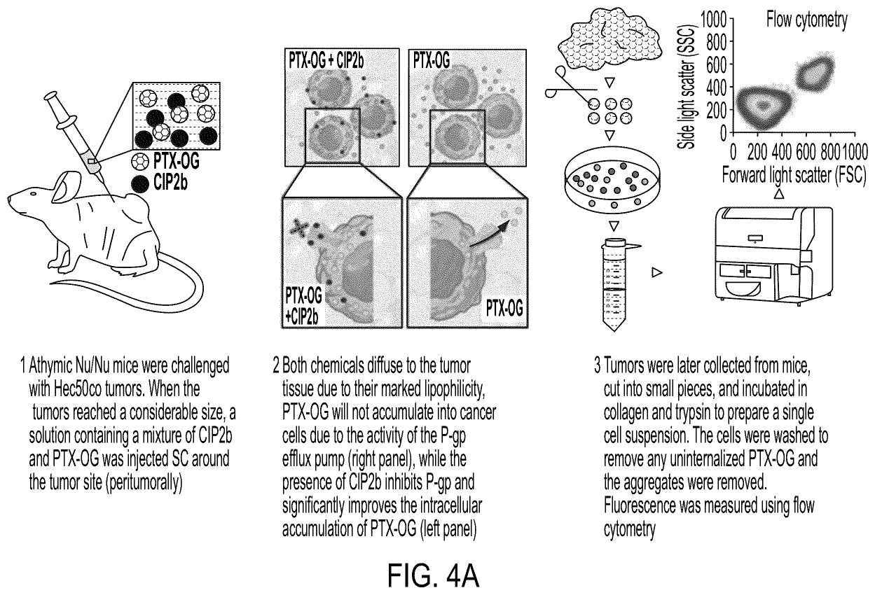

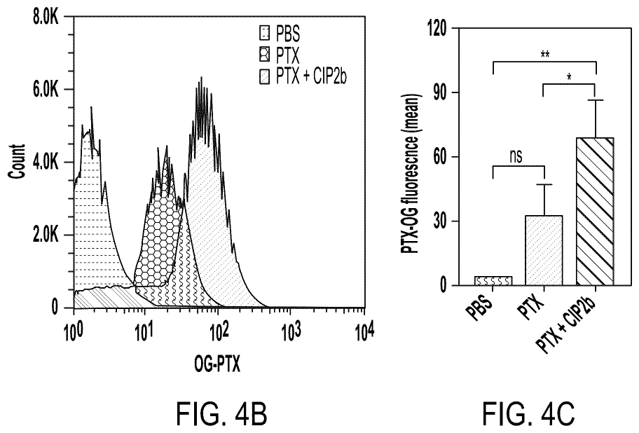

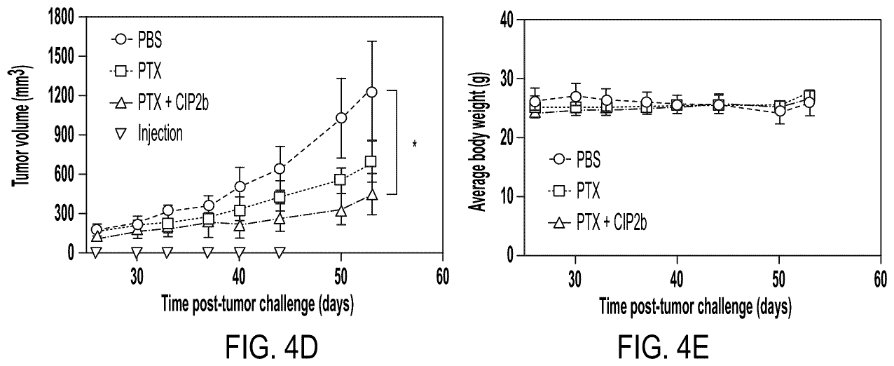

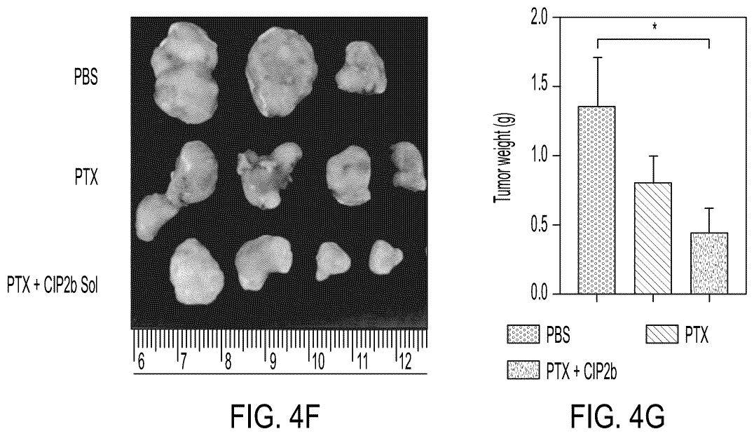

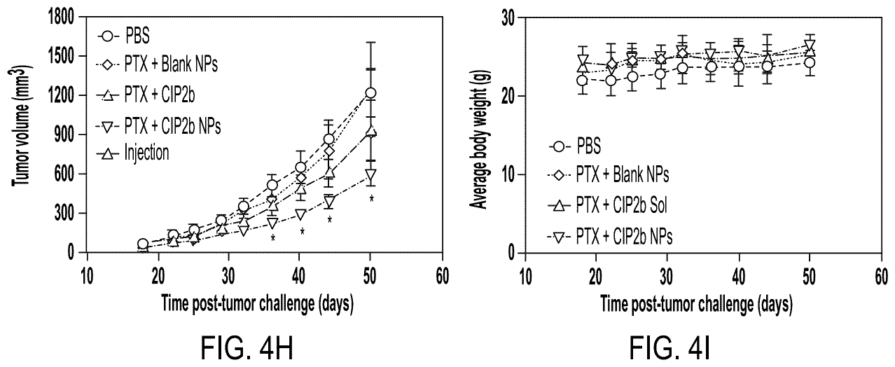

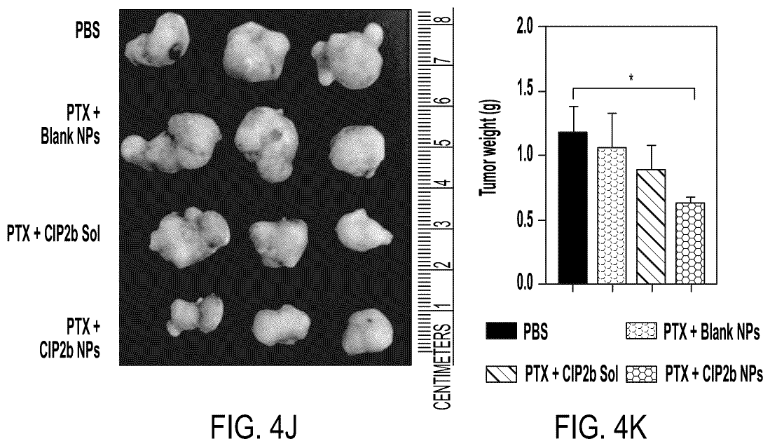

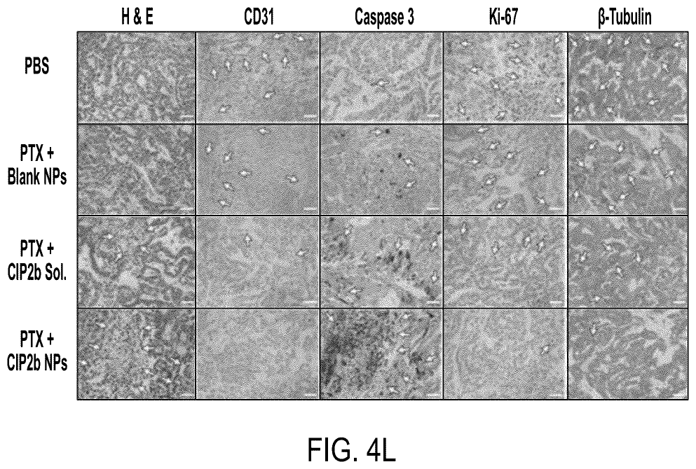

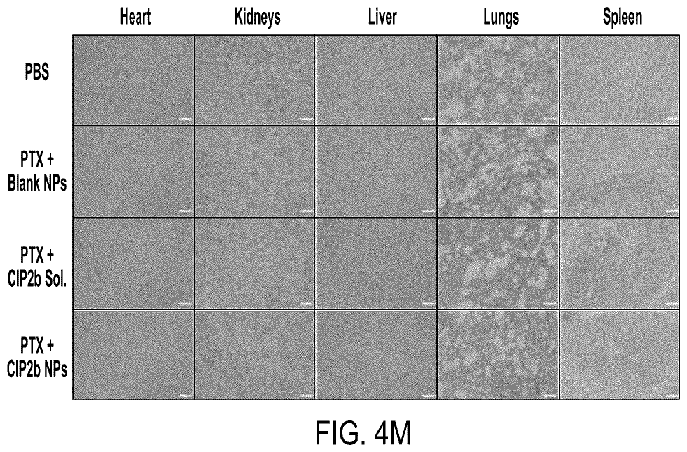

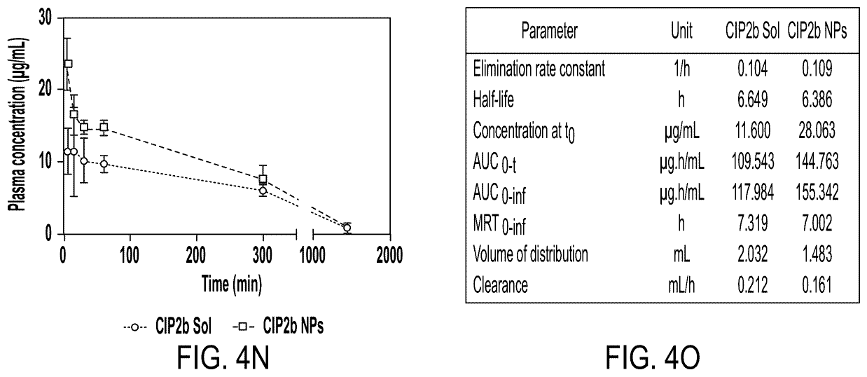

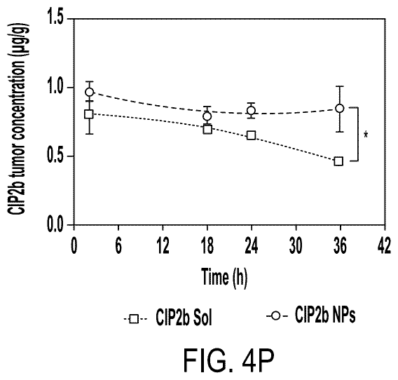

In vivo tumor accumulation of PTX The in vitro enhancement of intracellular accumulation of PTX as a result of co-administration with CIP2b was then tested in vivo. Following injection of mice bearing Hec50co tumors with PTX+CIP2b or PTX alone, tumors were made into single-cell suspensions, and all the media surrounding the tumor cells following dissociation (including extracellular matrix) were discarded to evaluate only the amount of PTX that were taken up inside cancer cells, and not that taken up inside the tumors without being able to get internalized by cells. The procedure carried out to perform this test is illustrated in FIG. 4A. The results revealed that mice treated with PTX alone exhibited marginal in vivo intracellular accumulation by tumor cells when compared to the untreated control mice. Interestingly, the co-administration of CIP2b with PTX significantly enhanced the intracellular accumulation of PTX within the tumor, causing a complete peak shift in the PTX histogram plot (FIG. 4B). The fluorescence means were almost doubled when CIP2b was added to PTX compared to PTX alone (p<0.05, FIG. 4C).

The co-administration of P-gp inhibitors with PTX may require the use of lower doses of the latter because the increased intracellular accumulation of PTX systemically may intensify adverse events. In a recent report, Stage et al. found that co-administration of a P-gp inhibitor (valspodar and verapamil) with PTX aggravates paclitaxel-induced peripheral neuropathy as PTX accumulates within neurons causing neuronal toxicity flowing inhibition of P-gp efflux in neurons (Stage T B, et. al., Clinical pharmacology and therapeutics. 2020; 108(3):671-80, doi: 10.1002/cpt.1847). This underscores the importance of using NPs with enhanced tumor accumulation to minimize collateral and non-specific inhibition of P-gp, as will be shown later.

Effect of CIP2b solution on the antitumor efficacy of PTX following peritumoral SC injection. In vivo antitumor efficacy study following the peritumoral SC injection in Hec50co tumor-bearing athymic Nu/Nu mice showed that the PBS group's tumors grew rapidly in comparison to the treated groups (FIG. 4D). Mice treated with PTX had average tumor volumes smaller than PBS control mice albeit not significantly (p>0.05, day 53 post-tumor challenge (last day)). In contrast, mice treated with the combination of PTX and CIP2b had the slowest tumor growth over time, and the average tumor volumes on day 53 post-tumor challenge were significantly smaller than that of the PBS group (455.2 vs. 1242.7 mm³, respectively, p<0.05). In addition, mice's body weights were monitored over time (FIG. 4E), and it was observed that the average body weight of treated mice was not different from the average body weight of mice injected with PBS. On the last day after mice euthanasia (i.e., day 53), tumors were collected and imaged (FIG. 4F), and their weights were also measured. It was found that the average weight of tumors of mice treated with the combination was significantly less than that of the untreated mice (p<0.05), while the average weight of tumors from mice treated with PTX did not show a significant difference against either the untreated group or the combination group (FIG. 4G).

The peritumoral injection was used in this experiment to test the proof of concept that whenever CIP2b and PTX co-exist in the tumor in sufficient quantities, the in vivo efficacy of PTX will be enhanced. However, this administration route is not clinically feasible. The clinically feasible options for a poorly soluble drug like CIP2b are limited, especially for cancer treatment where achieving as much intra-tumoral drug concentration as possible is a requirement. Surfactant/solubilizer-based vehicles (like Tween 80 and Cremophor EL) may not be the right answer, even though they are FDA-approved, because of a long history of vehicle-induced adverse events, including hypersensitivity reaction that may become fatal and necessitate pre-medication with corticosteroids. CIP2b is active in the micromolar range and this requires efficient intra-tumoral delivery, which led to the development and evaluation of CIP2b NPs.

Antitumor efficacy of PTX combined with CIP2b (solution vs NPs) following IV injection. In vivo efficacy study showed that IV injection of PTX+CIP2b solution in Hec50co tumor-bearing athymic Nu/Nu mice did not show a significant reduction of tumor volume compared to the PBS group (FIG. 4H) even though this combination was significantly effective when it was injected subcutaneously at the tumor site, while PTX alone was not (FIG. 4D and 4G). This leaves no doubt about the in vivo efficacy of this combination but underscores the compelling need for a delivery system with improved tumor accumulation. To our expectation, and in contrast to the moderate nonsignificant tumor growth inhibition displayed by PTX+CIP2b, mice treated with PTX+CIP2b NPs exhibited significant inhibition of tumor growth (FIG. 4H, p<0.05). The bodyweight of mice was also monitored and did not show any significant difference among all groups (FIG. 4I). Following mice euthanasia, tumors were harvested, and FIG. 4J shows a photograph of representative tumors from mice in each group. Furthermore, the average tumor weight of PTX+CIP2b NPs combination-treated mice was significantly less than that of the PBS-treated group (FIG. 4K).

Tumor histology and immunohistochemistry. H&E staining of tumors collected at the end of the efficacy study following IV injection of the treatments revealed that tumors obtained from mice treated with PTX+CIP2b NPs showed wide necrotic areas, and a massive abundance of pyknotic cells with condensed chromatin (pointed by yellow arrows, FIG. 4L) indicating necrosis and/or apoptosis (Elmore S A, D et. al., Toxicologic pathology. 2016; 44(2):173-88, doi: 10.1177/0192623315625859), compared to other treatment groups. This demonstrated that the proposed NP's-based combination treatment effectively induces cancer cell death inside tumors. Immunohistochemical staining of tumors collected from mice treated with PTX+CIP2b NPs further supported the antitumor efficacy outcomes. For example, anti-CD31 staining revealed the scarcity of vascular endothelial cells in the PTX+CIP2b NPs group, compared to other groups, especially the PBS group. In contrast to other tumor types like pancreatic tumors which are characterized by necrotic avascular areas in their centers (Naguib Y W, et. al., Neoplasia. 2016; 18(1):33-48, doi: 10.1016/j.neo.2015.11.012), these endometrial tumors are usually characterized by a high level of vascularization (Ebeid K, et. al.,Nature nanotechnology. 2018; 13(1):72-81, doi: 10.1038/s41565-017-0009-7), as evidenced by the abundance of vascular endothelial cells (FIG. 4L, PBS group). The absence of visible endothelia in the PTX+CIP2b NPs group indicates that this treatment may have inhibited neovascularization of tumors, which may require further investigation concerning the involvement of vascular endothelial growth factors (VEGF) pathways among other mechanisms by which this combination combat cancer cells. In addition, cleaved caspase 3 antibody stained tumor samples showed that tumors obtained from PTX+CIP2b NPs-treated mice involved extensive apoptosis in comparison to the other treatment groups while tumors from the PBS group showed negligible expression of caspase 3. Furthermore, staining the tumor tissues with Ki-67 antibody displayed markedly high cellular content of Ki-67 expression suggesting that PBS tumors had actively proliferating cells, in contrast to PTX+CIP2b NPs group, where Ki-67 expression was low, indicating that actively and rapidly proliferating cells are few. Moreover, PBS tumor samples stained with β-tubulin antibody demonstrated that the cells had high expression of β-tubulin whereas tumor cells in the PTX+CIP2b NPs group exhibited minimal expression of free unbound β-tubulin, suggesting that the drug combination efficiently targets tubulin and disrupts microtubule assembly.

Safety of the combination treatment following IV injection. On the last day of the previously mentioned experiment, serum was collected from mice following euthanasia and several toxicity parameters (liver enzymes, electrolytes, kidney function tests, etc.) were evaluated. No significant difference was recorded among all the treated groups in the parameters measured, indicating that no apparent toxicity was found. To confirm safety, vital organs of these mice were also collected following euthanasia and they were histologically examined following H&E staining. The absence of visible injury to the tissues (including apoptosis, karyorrhexis, pyknosis, or inflammatory cell invasion), or areas of necrosis in all treatment groups (FIG. 4M), in addition to the absence of any change of body weight (FIG. 4E and 41), indicates that the doses used were apparently safe. Even though the addition of CIP2b was expected to enhance the intracellular localization of PTX which may have caused increased toxicity, we did not observe any abnormality or tissue injury.

Pharmacokinetics. The study revealed that there were PK differences between CIP2b solution or CIP2b NPs following the IV injection as shown in the plasma concentration-time plots of CIP2b (FIG. 4N). It was found that the area under the curve (AUC 0-t) increased markedly when the CIP2b NPs (144.8 μg·h/mL) were used instead of the solution (109.5 μg·h/mL), while AUC 0-infinity increased to 155.3 vs 118.0, respectively (FIG. 4O). This increase in the AUC indicates that the presence of CIP2b inside the polymeric NPs provided protection of the molecule against metabolism. CIP2b is a lipophilic compound with a mean logP of 2.21 (data not shown), and thus it is difficult to inject it by IV route unless it is formulated in an aqueous-based vehicle or a suitable nano-carrier. The solubility of CIP2b was measured in different solubilizers using standard methods; it was found to have a relatively high solubility in Tween 80, Tween 20, PEG, and Cremophor EL. Since Tween 80 and ethanol are used clinically in the formulation of chemotherapeutics like docetaxel (Taxotere ®), Tween 80 was chosen to solubilize CIP2b to provide a control for in vivo comparisons.

CIP2b tumor accumulation following IV injection. Results showed that CIP2b levels in tumors were consistently higher and did not decline for 36 hours when CIP2b-loaded PLGA NPs were injected intravenously in Hec50co tumors-bearing mice (FIG. 4P). In contrast, tumors from mice injected IV with CIP2b solution (in Tween 80/ethanol/water) displayed a steep decline in their CIP2b level. This observation provides a clear answer to why the CIP2b solution was effective in combination with PTX when injected subcutaneously around the tumor but not as effective when injected intravenously. Similar results are commonly found in the literature (Ebeid K, et. al., Nature nanotechnology. 2018; 13(1):72-81, doi: 10.1038/s41565-017-0009-7; and Naguib Y W, et. al., Molecular pharmaceutics. 2014; 11(4):1239-49, doi: 10.1021/mp4006968) and can be directly explained by the EPR effect.

### Example 2. Biodistribution Study

### Methods

Female Balb-c mice (6-8 weeks, Jackson) were injected intravenously through the tail vein with CIP2b NPs (3.75 mg/kg) in PBS. After pre-determined time points (5, 15, 30, 60, 180, 240, and 1440 min), mice were euthanized by CO₂ followed by cervical dislocation, and their blood was collected by cardiac puncture in heparinized tubes. Vital organs (Heart, lungs, liver, spleen, pancreas, and kidneys) were collected from euthanized mice, and stored at −80° C. To extract the drug from the frozen organs, about 100-400 mg of thawed organs (on ice) were sliced homogenized using a bead tissue homogenizer (Fisher Brand Bead Mill-4, Hampton, NH) in screw capped 2-mL tubes containing about 25 zirconia beads and 0.25 mL of DPBS each. Two milliliters of ethyl acetate and 15 μL of the IS solution were added to about 400-700 mL of the tissue homogenate and CIP2b was extracted twice similar to the procedure mentioned above, and finally, 4 mL of ethyl acetate were evaporated to dryness under vacuum, and the residue was reconstituted as described in the pharmacokinetics study. Finally, the clear supernatant was injected into the HPLC for quantification. A standard curve was constructed for each organ using relevant blank mice organ homogenates spiked with 15 μL of CIP2b solutions with different concentrations and 15 μL of the IS solution. The standard curves were linear over a range of 0-25 μg/mL.

### Results and Discussion

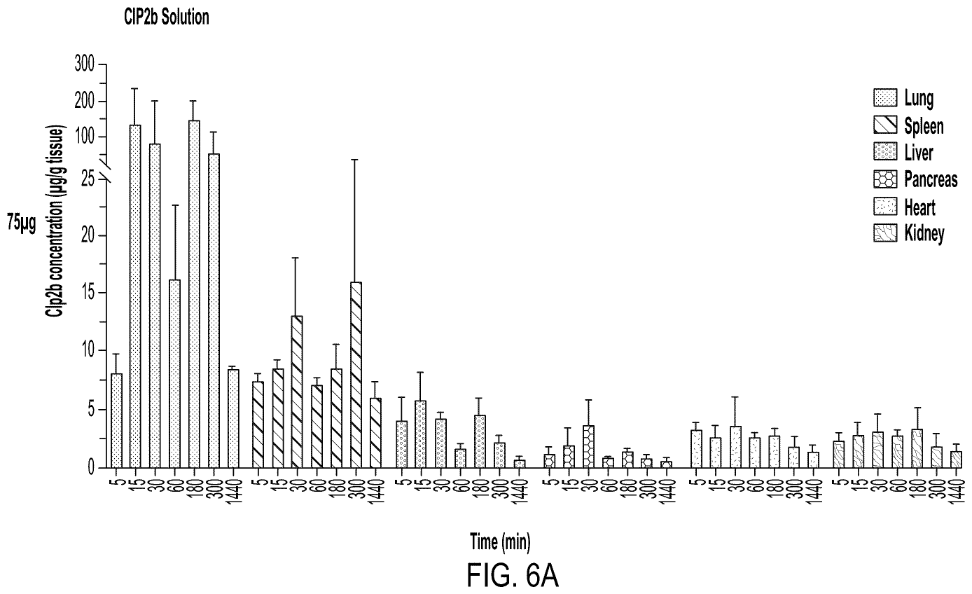

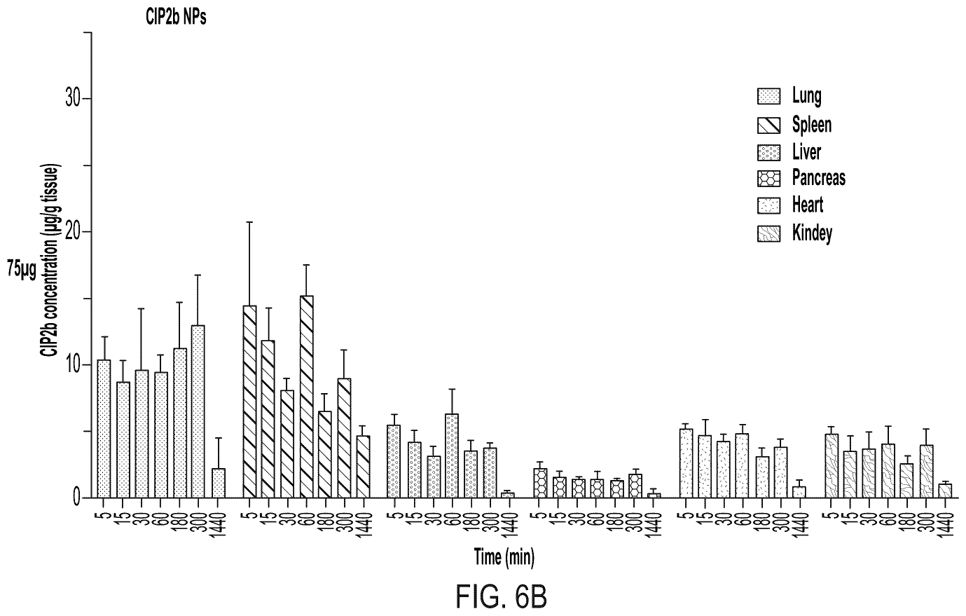

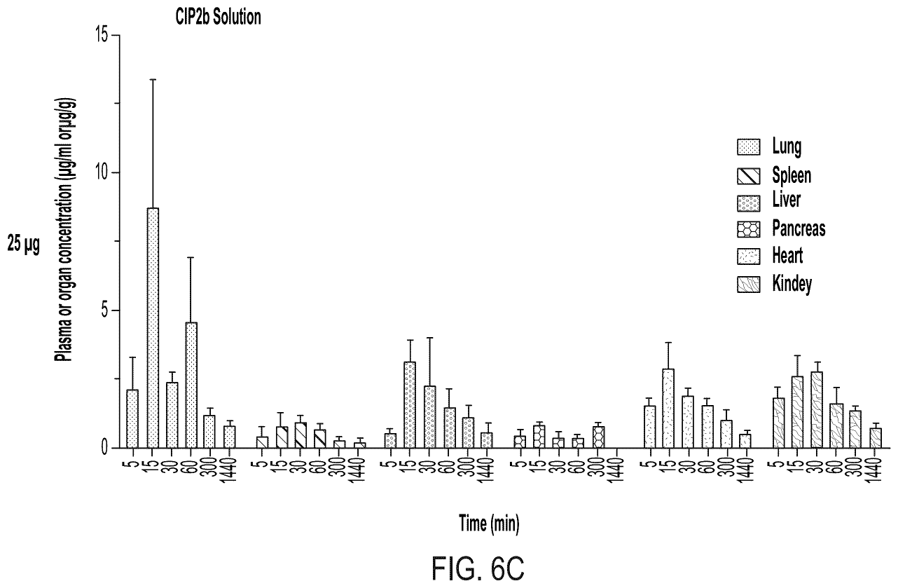

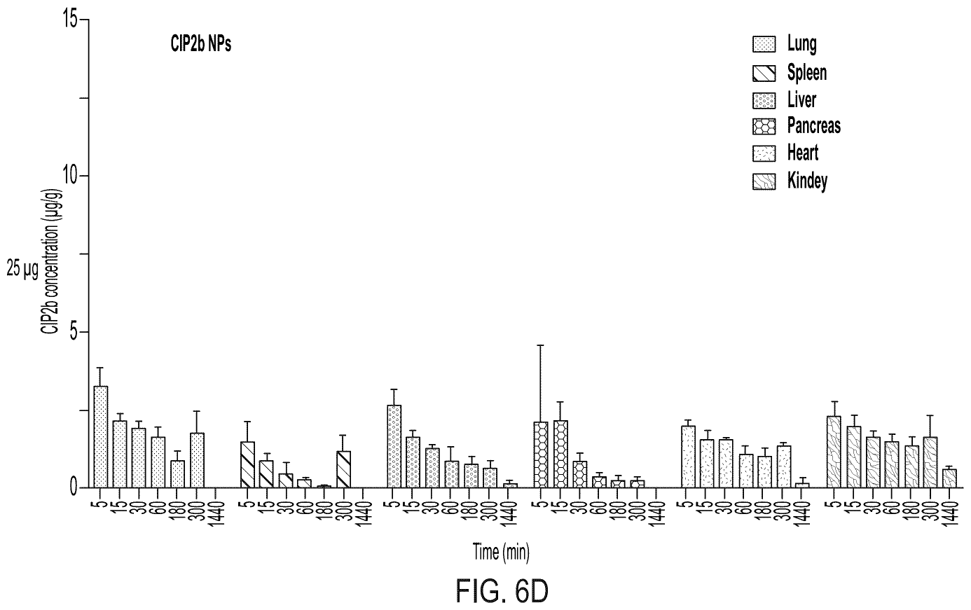

Results (FIG. 6) show that there is a tendency of CIP2b to substantially accumulate in the lungs either following the injection of a large dose, or a small dose, of CIP2b solution, compared to CIP2b NPs. When this is combined with the other findings that emphasize the ability of the NPs to improve the drug's tumor accumulation and plasma circulation time compared to the solution, it can be clearly seen that NPs modify the biodistribution profile of CIP2b towards better tumor accumulation, and less side effects in the lungs. This gains special importance based on the fact that PTX cytotoxicity is potentiated the most where CIP2b accumulates.

### Example 3. Cytotoxicity

These studies were performed to test the antitumor activity of the CIP2b/PTX drug combination against tumor cells such as colorectal cancer and breast cancer. In addition, these cytotoxicity assays were carried out to test a combination of CIP2b with other chemotherapies such as 5-fluorouracil (5-FU).

### Methods

Cytotoxicity was evaluated using the MTS assay described above.

### Results and Discussion

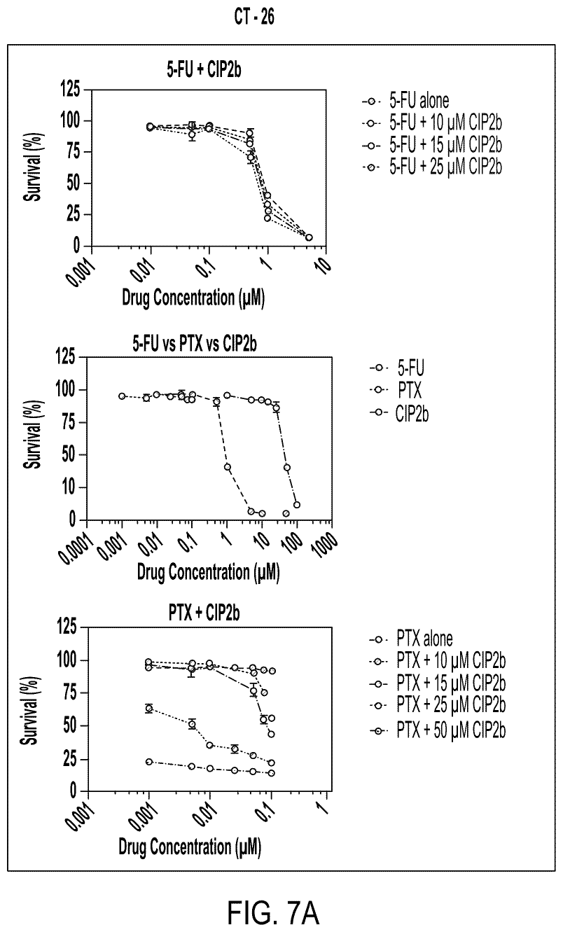

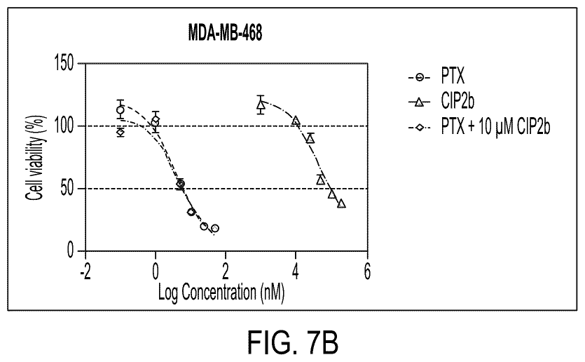

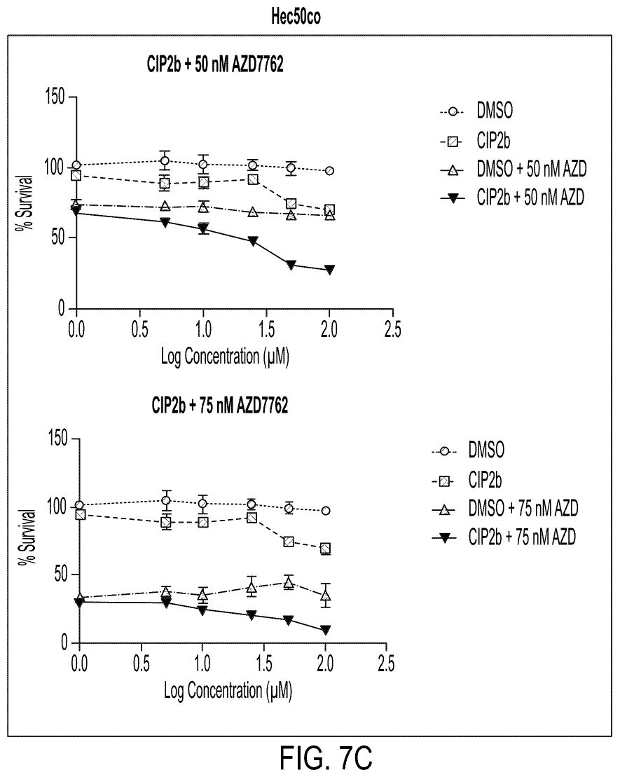

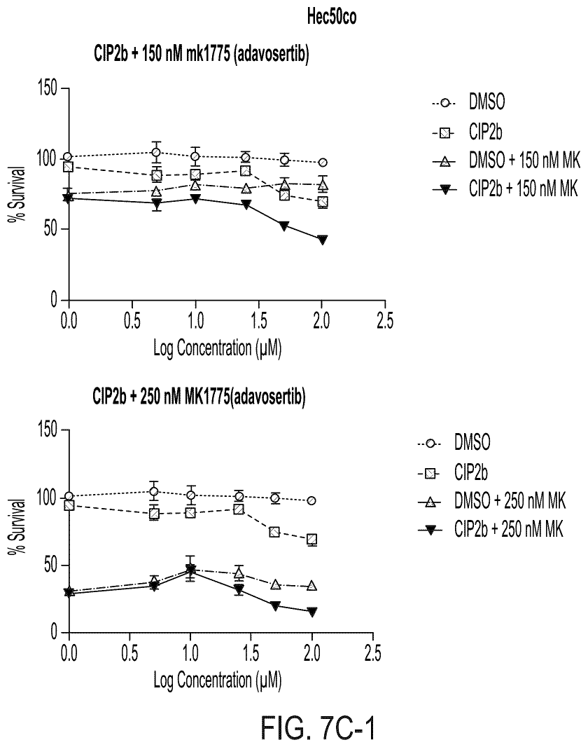

FIG. 7a shows that CIP2b substantially potentiates the activity of PTX against CT-26 murine colorectal cancer cells, while it does not potentiate the effect of 5-fluorouracil (5-FU). CT-26 are known to overexpress P-glycoprotein (P-gp) (Mercado-Lubo, R., et al., Nat Commun, 2016. 7: p. 12225; Park, J. W., et al., PLoS One, 2020. 15(2): p. e0228848; and Park, J. W., et al., Sci Rep, 2019. 9(1): p. 6462), and while PTX is a well-known substrate to P-gp as previously outlined, 5-FU is not (Choi, J. H., et al., Br J Cancer, 2002. 86(10): p. 1578-85). This may explain why CIP2b potentiated the efficacy of PTX in this cell line.

On the other hand, it is clear that CIP2b (10 μM) does not potentiate PTX efficacy in the triple-negative human breast cancer cell line MDA-MB-468 which itself is not known to overexpress P-gp (FIG. 7b), as Hoivik reported that the uptake of doxorubicin, a known P-gp substrate, into these cells was not influenced by the addition of the P-gp inhibitor verapamil (Hoivik, D., et al., Arch Biochem Biophys, 1997. 348(1): p. 174-82). Combinations of CIP2b with some checkpoint inhibitors like MK1775 and AZD7762 did not show any noticeable potentiation or synergism (FIG. 7c).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treating cancer in an animal, comprising administering paclitaxel or docetaxel and a quinolone of formula: 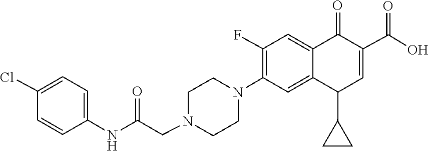 [SMILES: O=C(CN1CCN(c2cc3c(cc2F)C(=O)C(C(=O)O)=CC3C2CC2)CC1)Nc1ccc(Cl)cc1]
or a pharmaceutically acceptable salt thereof to the animal, wherein the cancer is selected from the group consisting of endometrial cancer, pancreatic cancer, colon cancer, and glioblastoma.

2. (canceled)

3. The method of claim 1, wherein the cancer is endometrial cancer.

4. The method of claim 1, wherein the animal is a human.

5. The method of claim 1, wherein the animal has been diagnosed with the endometrial cancer.

6-13. (canceled)

14. The method of claim 1, wherein the paclitaxel or docetaxel and the quinolone or the pharmaceutically acceptable salt thereof produce a synergistic cancer treating effect.

15. The method of claim 1, wherein a single formulation comprising the paclitaxel or docetaxel and the quinolone is administered to the animal.

16. The method of claim 1, wherein one formulation comprising the paclitaxel or docetaxel is administered to the animal and a second formulation comprising the quinolone or the pharmaceutically acceptable salt thereof is administered to the animal.

17. The method of claim 1, wherein the quinolone or the pharmaceutically acceptable salt thereof is formulated with PLGA nanoparticles.

18. A method comprising treating endometrial cancer in a human that has been diagnosed with endometrial cancer, by administering to the human a synergistic cancer treating amount of a combination of: a) paclitaxel or a pharmaceutically acceptable salt thereof, and 2) a compound of formula: 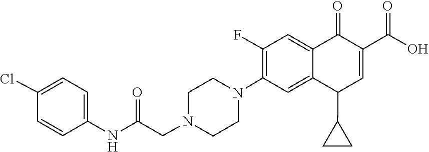 [SMILES: O=C(CN1CCN(c2cc3c(cc2F)C(=O)C(C(=O)O)=CC3C2CC2)CC1)Nc1ccc(Cl)cc1]
or a pharmaceutically acceptable salt thereof that is formulated with PLGA nanoparticles.

19. A composition comprising a paclitaxel or docetaxel and a quinolone of formula: 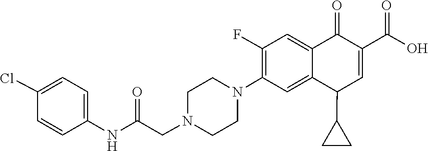 [SMILES: O=C(CN1CCN(c2cc3c(cc2F)C(=O)C(C(=O)O)=CC3C2CC2)CC1)Nc1ccc(Cl)cc1]
or a pharmaceutically acceptable salt thereof, and a pharmaceutically acceptable carrier.

20. (canceled)

21. A composition for treating endometrial cancer comprising, a synergistic endometrial cancer treating amount of a combination of: a) paclitaxel or a pharmaceutically acceptable salt thereof, 2) a compound of formula: 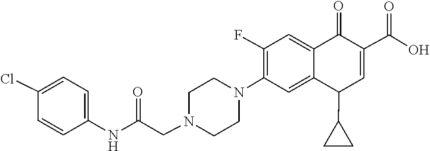 [SMILES: O=C(CN1CCN(c2cc3c(cc2F)C(=O)C(C(=O)O)=CC3C2CC2)CC1)Nc1ccc(Cl)cc1]
or a pharmaceutically acceptable salt thereof that is formulated with PLGA nanoparticles; and 3) a pharmaceutically acceptable carrier.
